(19) United States
(12) Patent Application Publication
(10) Pub. No.: US 2005/0154048 A1
(43) Pub. Date: Jul. 14, 2005

(54) Novel acyl hydrazino thiophene derivatives, process for preparing them, their use as medicinal products, pharmaceutical compositions and novel use

(75) Inventors: Didier Benard, Montsoult (FR); Jean-Francois Gourvest, Claye Souilly (FR)

Correspondence Address: ROSS J. OEHLER;AVENTIS PHARMACEUTICALS INC., ROUTE 202-206, MAIL CODE: D303A, BRIDGEWATER, NJ, 08807, US

(73) Assignee: Aventis Pharma S.A., Antony (FR)

(21) Appl. No.: 11/028,848

(22) Filed: Jan. 4, 2005

Related U.S. Application Data
(63) Continuation of application No. PCT/FR02/02335, filed on Jul. 4, 2002.

(51) Int. Cl. A61K 31/381; C07D 333/22

(52) U.S. Cl. 514/448; 549/72

(57) ABSTRACT

The invention concerns novel compounds of formula (I), 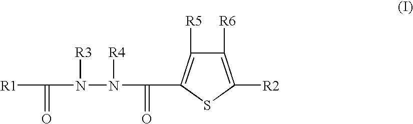 process for making, pharmaceutical compositions and methods of treating diseases associated with abnormal physiological behavior in the secretion and/or the activity of cysteine proteases especially cathepsins.

This application is a continuation of International Application No. PCT/FR2002/002335 filed Jul. 4, 2002.

The present invention relates to novel acyl hydrazino thiophene derivatives, to a process for preparing them, to the novel intermediates obtained, to their use as medicinal products, to pharmaceutical compositions containing them and to the novel use of such acyl hydrazino thiophene derivatives.

One subject of the invention is thus novel acyl hydrazino thiophene derivatives with inhibitory properties on metabolic enzymes. Such metabolic enzymes are especially kinases or proteases and especially cysteine proteases or serine proteases.

It may be recalled that it has been found that crustacean cysteine proteases induce in vertebrates biological effects identical to those of the hormone calcitonin, which suggests a common evolution of these enzymes and of this hormone: this finding is the basis of the development of novel treatments for diseases of cartilage and bone metabolism and particularly osteoporosis and Paget's disease.

Metabolic enzymes such as proteases or kinases are enzymes that are widely distributed in the animal kingdom. As nonexhaustive examples, bibliographic references that may be mentioned include the following documents for proteases: ‘Methods in Enzymology XLII (1975)’ and ‘Journal of Medicinal Chemistry’ vol. 43 No 3 (D. Leung, G. Abbenante and D. P. Fairlie) and the following document for kinases: ‘Methods in Enzymology, Vol 80 (1981) (Academic Press Inc.)’.

Among the proteases capable of selectively catalyzing the hydrolysis of polypeptide bonds, mention may be made of four main classes: aspartate proteases, serine proteases, metalloproteases and cysteine proteases.

Cysteine proteases are a family of proteases containing a thiol group in the active site. Such proteases exist in bacteria, viruses, eukaryotic microorganisms, plants and animals. Such cysteine proteases are very numerous, and mentioned hereinbelow, in a nonexhaustive manner, are examples of cysteine proteases such as cysteine proteases from plants, for instance papain, ficin, aleurain, orizain and actinidin; cysteine proteases from mammals, for instance cathepsins B, H, J, L, N, S, T, C, V, W, K or O, and O2, interleukin-converting enzyme (ICE), calpains I and II, and bleomycin hydrolase; viral cysteine proteases, for instance picornian 2A and 3C, aphthvirus, cardiovirus, comovirus, potyvirus I and II, adenovirus and togavirus endopeptidases, or alternatively polio or rhinovirus cysteine proteases. Cysteine proteases are also known as being essential for the life of certain parasites.

Among the cysteine proteases that may especially be mentioned are cathepsins and especially cathepsin K, B, L and S, and papain.

As documents whose teaching forms part of the present patent application, mention may be made of the following documents: WO 98/48799, WO 96/40737 and also Rawlings et al., Biochem. J. 290: 205-218 1993.

Such kinase or protease enzymes are involved in catabolization and inter- and intracellular communication processes: they play an important role in a large number of diseases of different fields, such as, especially, in the cardiovascular field, oncology, the central nervous system, inflammation, bone disorders, periodontal complaints and also parasitic, fungal or viral infectious diseases. This is why these proteins are targets of major interest for pharmaceutical research.

Inhibitors of such proteases may thus be useful in numerous and varied therapeutic fields.

The products of the present invention may thus be useful especially in preventing or treating diseases in which such metabolic enzymes are involved, for instance certain cardiovascular diseases, central nervous system diseases, inflammatory diseases, bone diseases, for instance osteoporosis, osteoarthritis or gingivitis, infectious diseases especially those requiring antiinfectious agents for their therapy, or certain cancers.

Such products of the present invention may thus be used especially for the treatment of diseases in which such proteases are involved, and especially diseases associated with substantial loss of bone or cartilage, as defined hereinbelow.

Bone tissue is the site of permanent continuous remodeling that ensures phosphocalcic homeostasis and maintains the mechanical qualities of the bones. Whatever the nature of the bone (long, short, flat, etc.), the modeling takes place according to a sequence of four successive phases:
1. Activation: comprises differentiation of the pre-osteoclasts by means of hormones or physical signals;
2. Bone resorption: performed by the mature osteoclasts, commencing with adhesion of these cells to the bone matrix, resulting in the organization of the apical membrane into a sealing zone delimiting a microenvironment (Howship's lacunae) into which are released protons produced by the cell, responsible for demineralization of the mineral part of the bones, and also proteolytic enzymes released across the membrane, responsible for the degradation of the matrix proteins;
3. Inversion: characterized by detachment of the osteoclasts, which are replaced with osteoblasts;
4. Reconstruction: neoformation of the matrix (osteoid) and mineralization, performed by the osteoblasts.

The duration of a remodeling cycle in a normal adult human is about three months.

Several proteolytic enzymes are involved in the degradation of matrix proteins. This is the case especially for cathepsins K, B, L and S and also for certain metalloproteases.

The cathepsins are defined as being lysosomal proteins, the majority of which are “papain-like” enzymes. Several sequences of the human genome coding for cysteine cathepsins have been identified to date (e.g.: B, H, L, S, C, K, O, F, V, X, W). All these enzymes contain a cysteine-histidine-asparagine pair in their catalytic site, forming a thiolate-imidazolium pair required for the enzymatic activity. They are capable of hydrolyzing various substrates, for instance peptides, amides, esters, thiol esters and thiono esters.

The family of cysteine cathepsins comprises various proteolytic enzymes involved in several physiological processes, and may thus play important roles in various pathologies.

Cathepsin K (previously known as O or O2) was identified from a rabbit cDNA library, and then localized in human ovaries and osteoclasts (in the adherent mature cells (Tezuka K, Tezuka Y, Maejima S: J. Biol. Chem. (1994) 269: 1106-1109)). It is expressed predominantly in said cells (Brömme D, Okamoto K, Wang B, Biroc S: J. Biol. Chem. (1996) 271: 2126-2132) and is thus considered as being the key protease of this process, with respect to its localization (Yamaza T, Goto T, Kamiya T, Kobayashi Y, Sakai H, Tanaka T: Bone. (1998) 23: 499-509) and since it is one of the few proteases whose natural substrate is native collagen, constituting 90% of the proteins of bone matrix (Garnero P, Borel 0, Byrjalsen I, Ferreras M, Drake F H, McQueney M S, Foged N T, Delmas P, Delaissé J M: J. Biol.

Chem. (1998) 273: 32347-32352); the remaining 10% consisting of numerous noncollagen proteins that are occasionally substrates of cathepsin K (osteopontin, thrombospondin, fibronectin and vitronectin), and occasionally regulators of proteolytic activity (proteoglycans).

Transgenic mice, KO for the gene coding for cathepsin K, have an osteopetrotic phenotype, including an increase in bone mass and thick bones of poor quality. In this model, bone resorption is virtually absent. Only demineralization remains. This phenotype compares with that of patients suffering from pycnodysostosis (Toulouse-Lautrec disease), a genetic disease resulting in the production of inactive cathepsin K, which is responsible for facial hypoplasia and the premature arrestation of growth of the long bones with severe osteosclerosis (Saftig P, Hunziker E, Wehmeyer O, Jones S, Boyde A, Rommerskirch W, Moritz J D, Schu P, Von Figura K: Proc. Natl. Acad. Sci. USA (1998) 95: 13453-13458).

The use of cathepsin K antisense oligonucleotides (S—ODN) has made it possible to inhibit the osteoclast-mediated bone resorption in a test of “in vitro” pit formation (Inuit F W, J. Biol. Chem (1997) 272: 8109-8112).

The involvement of cathepsin K in the process of bone resorption makes this enzyme a favored target for treating pathologies, such as osteoporosis, caused by an imbalance in bone turnover in favor of resorption, the target for novel molecules for inhibiting enzymatic activity.

The present invention thus relates to novel acyl hydrazino thiophene derivatives that are inhibitors of cysteine proteases, more particularly of the family of cathepsins B and K, and especially of the family of cathepsin K.

One subject of the present invention is thus the products of formula (I): 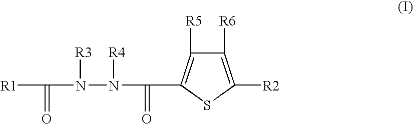
in which:
R1 represents a linear or branched alkyl or alkoxy radical containing up to 8 carbon atoms substituted either with a monocyclic radical or consisting of carbocyclic or heterocyclic, saturated or unsaturated fused rings that are up to 14-membered, containing one or more identical or different hetero atoms, O, N, NH or S, such a monocyclic radical or a radical consisting of fused rings itself being optionally substituted as indicated below,
or with an amino radical that is itself optionally substituted with a radical —C(O)—O—R7 or —C(O)—R7,
R2 represents:
a) a linear or branched alkyl radical containing up to 8 carbon atoms optionally substituted with one or more radicals chosen from the radicals —NH—C(O)—O—R7, radicals —NH—C(O)-alk-NH—C(O)—O—R7 and monocyclic radicals or radicals consisting of carbocyclic or heterocyclic, saturated or unsaturated fused rings that are up to 14-membered, containing one or more identical or different hetero atoms, O, N, NH or S, such monocyclic radicals or radicals consisting of fused rings, themselves being optionally substituted as indicated below,
b) a phenyl radical optionally substituted with a linear or branched alkoxy radical containing up to 6 carbon atoms or an arylalkoxy radical, these alkoxy and arylalkoxy radicals themselves being optionally substituted as indicated below,
c) a naphthyl radical optionally substituted as indicated below,
d) an amino radical optionally substituted with one or two radicals, which may be identical or different, chosen from linear or branched alkyl radicals containing up to 6 carbon atoms, radicals —C(O)-alk-NH—C(O)—O—R7 and arylalkyl radicals, themselves optionally substituted with an alkoxyphenoxy radical in which the linear or branched alkoxy radical contains up to 6 carbon atoms, these alkyl, arylalkyl, alkoxy and alkoxyphenoxy radicals themselves being optionally substituted as indicated below,
e) >=N—O—R8,
R3 and R4, which may be identical or different, represent a hydrogen atom or an alkyl, arylalkyl or aryl radical, in which the alkyl radicals are linear or branched, containing up to 6 carbon atoms, and the aryl radicals contain up to 10 carbon atoms, these alkyl, arylalkyl and aryl radicals being optionally substituted with a halogen atom, a hydroxyl radical or a linear or branched alkyl or alkoxy radical containing up to 4 carbon atoms,
R5 and R6, which may be identical or different, are chosen from a hydrogen atom, a hydroxyl radical and linear or branched alkyl and alkoxy radicals containing up to 6 carbon atoms, these alkyl and alkoxy radicals themselves being optionally substituted as indicated below,
R7 represents a linear or branched alkyl radical containing up to 6 carbon atoms, optionally substituted with an optionally substituted aryl or heteroaryl (indazolyl) radical,
R8 represents a hydrogen atom or a linear or branched alkyl radical containing up to 6 carbon atoms optionally substituted with an aryl (phenyl) radical, it being understood that all alkyl and alkoxy radicals, monocyclic radicals or radicals consisting of fused rings, aryl, heteroaryl, arylalkyl, arylalkoxy and alkoxyphenoxy radicals indicated above as being optionally substituted, are optionally substituted with one or more radicals chosen from halogen atoms; hydroxyl radicals; alkyl, alkenyl, alkylthio or alkoxy containing up to 6 carbon atoms; up to 6-membered cycloalkyl; acyl containing up to 7 carbon atoms; cyano; nitro; free, salified or esterified carboxyl; tetrazolyl; indazolyl; —NH2, —NH(alk) or —N(alk)(alk); —SO2-NH—CO—NHR5 in which R5 represents alk or phenyl; —C(O)—NH2, —C(O)—NH(alk), —C(O)—N(alk)(alk), —NH—C(O)-(alk), —N(alk)-C(O)-alk; and phenyl itself optionally substituted with an alkyl or alkoxy radical containing up to 4 carbon atoms,
it being understood that alk represents a linear or branched alkyl radical containing up to 4 carbon atoms, said products of formula (I) being in all the possible racemic, enantiomeric and diastereoisomeric isomer forms, and also the addition salts with mineral and organic acids or with mineral and organic bases of said products of formula (I).

In the products of formula (I) and in the text hereinbelow:
the term “linear or branched alkyl radical” denotes methyl, ethyl, propyl, isopropyl, butyl, isobutyl, sec-butyl, tert-butyl, pentyl, isopentyl, hexyl and isohexyl radicals, and also heptyl, octyl, nonyl and decyl radicals, and also the linear or branched positional isomers thereof,
the term “linear or branched alkoxy radical” denotes methoxy, ethoxy, propoxy, isopropoxy, linear, secondary or tertiary butoxy, pentoxy or hexoxy, and also the linear or branched positional isomers thereof,
the term “halogen atom” denotes chlorine, bromine, iodine or fluorine atoms, and preferably a chlorine, bromine or fluorine atom,
the term “carbocyclic or heterocyclic monocyclic radical or radical consisting of saturated or unsaturated fused rings that are up to 14-membered, containing one or more identical or different hetero atoms chosen from O, N, NH and S” includes the following definitions:
the term “saturated carbocyclic radical” especially denotes a cycloalkyl radical,
the term “cycloalkyl radical” denotes cyclopropyl, cyclobutyl, cyclopentyl and cyclohexyl radicals, and most particularly cyclopentyl and cyclohexyl radicals,
the term “monocyclic heterocyclic radical” denotes a saturated or unsaturated radical, which is especially 5- or 6-membered such that one or more of the ring members represents an oxygen, sulfur or nitrogen atom: such a heterocyclic radical thus denotes a carbocyclic radical interrupted with one or more hetero atoms chosen from oxygen, nitrogen and sulfur atoms, it being understood that the heterocyclic radicals may contain one or more hetero atoms chosen from oxygen, nitrogen and sulfur atoms, and that when these heterocyclic radicals comprise more than one hetero atom, the hetero atoms of these heterocyclic radicals may be identical or different. Mention may be made especially of the following radicals: dioxolane, dioxane, dithiolane, thiooxolane, thiooxane, morpholinyl, piperazinyl, piperazinyl substituted with a linear or branched alkyl radical containing up to 4 carbon atoms, piperidyl, thienyl such as 2-thienyl and 3-thienyl, furyl such as 2-furyl, pyrimidinyl, pyridyl such as 2-pyridyl, 3-pyridyl and 4-pyridyl, pyrimidyl, pyrazolinyl, pyrrolyl, thiazolyl, isothiazolyl, diazolyl, thiadiazolyl, free or salified triazolyl, thiadiazolyl, thiatriazolyl, oxazolyl, oxadiazolyl, 3- or 4-isoxazolyl. Mention may be made most particularly of morpholinyl, thienyl such as 2-thienyl and 3-thienyl, furyl such as 2-furyl, tetrahydrofuryl, thienyl, tetrahydrothienyl, pyrrolyl, pyrrolinyl, pyrazolinyl, isoxazolyl, pyridyl and pyrrolidinyl radicals,
the term “heterocyclic radical consisting of fused rings” denotes a saturated or unsaturated radical that is up to 14-membered such that one or more of the ring members represents an oxygen, sulfur or nitrogen atom, and especially fused heterocyclic groups containing at least one hetero atom chosen from sulfur, nitrogen and oxygen, for example benzothienyl such as 3-benzothienyl, benzothiazolyl, quinolyl, isoquinolyl, tetralone, benzofuryl, benzopyrrolyl, benzimidazolyl, benzoxazolyl, thionaphthyl, indolyl or purinyl,
the term “aryl” denotes monocyclic radicals or radicals consisting of unsaturated carbocyclic fused rings as defined above and also especially denotes phenyl and naphthyl radicals, especially the phenyl radical,
the term “heteroaryl” denotes monocyclic radicals or radicals consisting of unsaturated heterocyclic fused rings as defined above and thus especially denotes indazolyl, thienyl, benzothienyl, furyl, pyranyl, isobenzofuryl, pyrrolyl, imidazolyl, pyrazolyl, pyridyl, pyrazinyl, pyrimidinyl, pyridazinyl, indolyl and its positional isomers of the nitrogen atom, indazolyl, quinolyl and its positional isomers of the nitrogen atom, quinazolinyl and its positional isomers of the two nitrogen atoms, isothiazolyl, isoxazolyl and furazanyl radicals,
the term “arylalkoxy” denotes radicals in which aryl and alkoxy have the definitions given above and especially represents phenylalkoxy radicals in which alkoxy contains up to 4 carbon atoms,
the term “alkoxyphenoxy” denotes radicals in which alkoxy has the definition given above and especially represents alkoxy containing up to 4 carbon atoms,
the term “arylalkyl” denotes radicals in which aryl and alkyl have the definitions given above and especially represents phenylalkyl radicals in which alkyl contains up to 4 carbon atoms,
the term “alkylphenyl” denotes a phenyl radical substituted with one or more linear or branched alkyl radicals as defined above, preferably containing up to 4 carbon atoms,
the terms “NH(alk)” and “N(alk)(alk)” denote an amino radical substituted, respectively, with one or two alkyl radicals, such alkyl or alk radicals being linear or branched and preferably containing up to 4 carbon atoms,
the term “acylamino” denotes radicals —C(O)—NH2, —C(O)—NH(alk) and —C(O)—N(alk)(alk): in these radicals and also in the radicals NH—C(O)-(alk), —N(alk)-C(O)-(alk)- and —C(O)-alk-NH—C(O)—O—R7, the terms alk, NH(alk) and N(alk)(alk) have the meanings given above,
the term “acyl” denotes a radical R—C(O)— in which R represents a radical chosen from a hydrogen atom, linear or branched alkyl radicals containing up to 6 carbon atoms, a phenyl radical or a pyrrolidinyl radical: the term “acyl” thus especially denotes formyl radicals and acetyl, propionyl, butanoyl, pentanoyl, hexanoyl, benzoyl and pyrrolidinylcarbonyl radicals,
the term “alkenyl” denotes linear or branched radicals containing up to 6 carbon atoms: mention may be made especially of vinyl, 1-propenyl, allyl, butenyl and 3-methyl-2-butenyl radicals,
the term “alkylthio” denotes linear or branched radicals containing up to 6 carbon atoms, especially such as methylthio, ethylthio, propylthio, isopropylthio, butylthio, isobutylthio, sec-butylthio, tertbutylthio, pentylthio, isopentylthio, hexylthio or isohexylthio radicals, and also the linear or branched positional isomers thereof: among these alkylthio radicals, of those mentioned above, radicals containing up to 4 carbon atoms are preferably chosen,
the term “amino acid” denotes the “natural” amino acids or the “unnatural” amino acids known to those skilled in the art, which may be found in standard documents such as, for example, the document ‘Amino Acids’ (1999), 16 (3-4), 345-379.

The carboxyl radical(s) of the products of formula (I) may be salified or esterified with various groups known to those skilled in the art, among which mention may be made, for example, of:
among the salification compounds, mineral bases such as, for example, one equivalent of sodium, potassium, lithium, calcium, magnesium or ammonium, or organic bases such as, for example, methylamine, propylamine, trimethylamine, diethylamine, triethylamine, N,N-dimethylethanolamine, tris(hydroxymethyl)aminomethane, ethanolamine, pyridine, picoline, dicyclohexylamine, morpholine, benzylamine, procaine, lysine, arginine, histidine or N-methylglucamine,
among the esterification compounds, alkyl radicals to form alkoxycarbonyl groups such as, for example, methoxycarbonyl, ethoxycarbonyl, tert-butoxycarbonyl or benzyloxycarbonyl, these alkyl radicals possibly being substituted with radicals chosen, for example, from halogen atoms and hydroxyl, alkoxy, acyl, acyloxy, alkylthio, amino or aryl radicals, such as, for example, in the chloromethyl, hydroxypropyl, methoxymethyl, propionyloxymethyl, methylthiomethyl, dimethylaminoethyl, benzyl or phenethyl groups.

The addition salts with mineral or organic acids of the products of formula (I) may be, for example, the salts formed with hydrochloric acid, hydrobromic acid, hydriodic acid, nitric acid, sulfuric acid, phosphoric acid, propionic acid, acetic acid, trifluoroacetic acid, formic acid, benzoic acid, maleic acid, fumaric acid, succinic acid, tartaric acid, citric acid, oxalic acid, glyoxylic acid, aspartic acid or ascorbic acid, alkylmonosulfonic acids, for instance methanesulfonic acid, ethanesulfonic acid or propanesulfonic acid, alkyldisulfonic acids, for instance methanedisulfonic acid or α,β-ethanedisulfonic acid, arylmonosulfonic acids such as benzenesulfonic acid, and aryldisulfonic acids.

It may be recalled that stereoisomerism may be defined in its broad sense as being the isomerism of compounds having the same structural formulae, but whose various groups are arranged differently in space, such as, especially, in monosubstituted cyclohexanes in which the substituent may be in an axial or equatorial position, and the various possible rotational conformations of ethane derivatives. However, there is another type of stereoisomerism, due to the different spatial arrangements of attached substituents, either on double bonds or on rings, which is often referred to as geometrical isomerism or cis-trans isomerism. The term “stereoisomers” is used in the present patent application in its broadest sense and thus concerns all of the compounds indicated above.

It may be noted that when R1 represents alkyl substituted with amino which is itself optionally substituted, R1 especially represents an optionally substituted decarboxylated amino acid residue and more specifically an optionally substituted decarboxylated Leu residue.

One subject of the present invention is thus the products of formula (I) as defined above corresponding to formula (Ia): 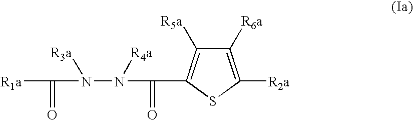
in which:
R1a represents a linear or branched alkyl or alkoxy radical containing up to 8 carbon atoms substituted with a phenyl radical, a radical consisting of saturated or unsaturated carbocyclic fused rings that are up to 14-membered and an amino radical that is itself optionally substituted with a radical —C(O)—O—R7a or —C(O)—R7a, this phenyl radical and these radicals consisting of saturated or unsaturated carbocyclic fused rings being optionally substituted as indicated below,
R2a represents:
a) a linear or branched alkyl radical containing up to 6 carbon atoms optionally substituted with one or more radicals chosen from the radicals NH—C(O)—O—R7a and —NH—C(O)-alk-NH—C(O)—O—R7, a phenyl radical and saturated or unsaturated heterocyclic radicals that are up to 10-membered,
b) a phenyl radical optionally substituted with a linear or branched alkoxy radical containing up to 6 carbon atoms or a benzyloxy radical,
c) a naphthyl radical,
d) an amino radical optionally substituted with one or two radicals, which may be identical or different, chosen from linear or branched alkyl radicals containing up to 4 carbon atoms, radicals —C(O)-alk-NH—C(O)—O—R7a and a benzyl radical, itself optionally substituted with an alkoxyphenoxy radical in which the alkoxy radical contains up to 4 carbon atoms,
e) >=N—O—R8a,
R3a and R4a, which may be identical or different, represent a hydrogen atom, a linear or branched alkyl radical containing up to 6 carbon atoms or a phenyl radical optionally substituted with a hydroxyl or alkoxy radical containing up to 4 carbon atoms,
R5a and R6a, which may be identical or different, are chosen from the hydrogen atom, the hydroxyl radical and linear or branched alkyl or alkoxy radicals containing up to 6 carbon atoms optionally substituted with a phenyl or indazolyl radical,
R7a represents a linear or branched alkyl radical containing up to 4 carbon atoms optionally substituted with a phenyl radical or an indazolyl radical,
R8a represents a hydrogen atom or a linear or branched alkyl radical containing up to 4 carbon atoms optionally substituted with a phenyl radical,
it being understood that alk represents a linear or branched alkyl radical containing up to 4 carbon atoms, said products of formula (Ia) being in any possible racemic, enantiomeric or diastereoisomeric isomer form, and also the addition salts with mineral and organic acids or with mineral and organic bases of said products of formula (Ia).

A subject of the present invention is more particularly the products of formula (I) as defined above corresponding to formula (Ib): 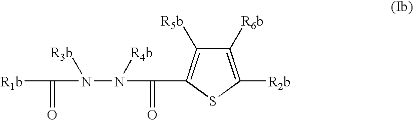
in which:
R1b represents a linear or branched alkyl or alkoxy radical containing up to 6 carbon atoms substituted with a radical chosen from phenyl and fluorene (Ex. 1 and 20) radicals and an amino radical that is itself optionally substituted with a radical —C(O)—O—R7b or —C(O)—R7b,
R2b represents:
a) a linear or branched alkyl radical containing up to 6 carbon atoms optionally substituted with one or more radicals chosen from the radicals —NH—C(O)—O—R7b and —NH—C(O)-alk-NH—C(O)—O—R7b and phenyl, pyridyl, pyridyl, piperidyl, benzofuryl, morpholinyl, quinolyl, indolyl, benzimidazolyl and indazolyl radicals,
b) a phenyl radical optionally substituted with a linear or branched alkoxy radical containing up to 4 carbon atoms or a benzyloxy radical,
c) a naphthyl radical,
d) an amino radical optionally substituted with one or two radicals, which may be identical or different, chosen from linear or branched alkyl radicals containing up to 4 carbon atoms, radicals —C(O)-alk-NH—C(O)—O-benzyl and a benzyl radical, itself optionally substituted with an alkoxyphenoxy radical in which the alkoxy radical contains up to 4 carbon atoms,
e) >=N—O—R8b,
R3b and R4b represent a hydrogen atom,
R5b and R6b, which may be identical or different, represent a hydrogen atom, a hydroxyl radical, a linear or branched alkyl or alkoxy radical containing up to 4 carbon atoms optionally substituted with a phenyl radical,
R7b represents a linear or branched alkyl radical containing up to 4 carbon atoms optionally substituted with a phenyl radical or an indazolyl radical,
R8b represents a hydrogen atom or a benzyl radical,
it being understood that alk represents a linear or branched alkyl radical containing up to 4 carbon atoms, said products of formula (Ib) being in any possible racemic, enantiomeric or diastereoisomeric isomer form, and also the addition salts with mineral and organic acids or with mineral and organic bases of said products of formula (Ib).

A subject of the present invention is most particularly the products of formulae (I), (Ia) and (Ib) as defined above, in which R3, R3a, R4, R4a, R5, R5a, R5b, R6, R6a and R6b represent a hydrogen atom,
said products of formulae (I), (Ia) and (Ib) being in any possible racemic, enantiomeric or diastereoisomeric isomer form, and also the addition salts with mineral and organic acids or with mineral and organic bases of said products of formulae (I), (Ia) and (Ib).

A subject of the present invention is even more particularly the products of formula (I) as defined above, corresponding to the following formulae:
5-[(2S)-1-[[4-methyl-1-oxo-2-[[(phenylmethoxy)-carbonyl]amino]pentyl]amino]ethyl]-2-thiophenecarboxylic acid 2-[(2S)-4-methyl-1-oxo-2-[[(phenylmethoxy)carbonyl]amino]pentyl]hydrazide
5-[1-(hydroxyimino)ethyl]-2-thiophenecarboxylic acid 2-[[(9H-fluoren-9-yl)methoxy]carbonyl]hydrazide
5-[2-(phenylmethoxy)phenyl]-2-thiophenecarboxylic acid 2-[(2S)-4-methyl-1-oxo-2-[[(phenylmethoxy)-carbonyl]amino]pentyl]hydrazide
5-[[[3-(4-methoxyphenoxy)phenyl]methyl](phenylmethyl)amino]-2-thiophenecarboxylic acid 2-[(2S)-4-methyl-1-oxo-2-[[(phenylmethoxy)carbonyl]amino]pentyl]-hydrazide
5-[1-[(phenylmethoxy)imino]ethyl]-2-thiophenecarboxylic acid 2-[(2S)-4-methyl-1-oxo-2-[[(phenylmethoxy)carbonyl]amino]pentyl]hydrazide
5-[1-[[(phenylmethoxy)carbonyl]amino]ethyl]-2-thiophenecarboxylic acid 2-[[(9H-fluoren-9-yl)methoxy]-carbonyl]hydrazide
5-[1-[[(phenylmethoxy)carbonyl]amino]ethyl]-2-thiophenecarboxylic acid 2-[(2S)-4-methyl-1-oxo-2-[[(phenylmethoxy)carbonyl]amino]pentyl]hydrazide
5-[3-methyl-1-[(phenylmethoxy)carbonyl]amino]butyl]-2-thiophenecarboxylic acid 2-[(2S)-4-methyl-1-oxo-2-[[(phenylmethoxy)carbonyl]amino]pentyl]hydrazide
5-[3-methyl-1-[[(phenylmethoxy)carbonyl]amino]butyl]-2-thiophenecarboxylic acid 2-[(2S)-4-methyl-1-oxo-2-[[(phenylmethoxy)carbonyl]amino]pentyl]hydrazide isomer A
5-[3-methyl-1-[[(phenylmethoxy)carbonyl]amino]butyl]-2-thiophenecarboxylic acid [(2S)-4-methyl-1-oxo-2-[[(phenylmethoxy)carbonyl]amino]pentyl]hydrazide isomer B.

A subject of the present invention is also a process for preparing the products of formula (I) as defined above, characterized in that the starting material used is a compound of formula (II): 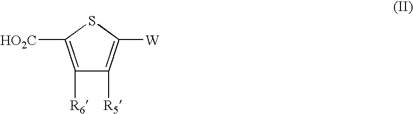
in which W represents a hydrogen atom, a halogen atom or a nitro radical, R5′ and R6′ have the meanings given above, respectively, for R5 and R6, in which the optional reactive functions are optionally protected with protecting groups,
which product of formula (II), depending on the value of W, may be subjected to the following reactions:
a) when W represents a hydrogen atom, the product of formula (II) may be subjected to the reactions of routes i) or ii):
route i) the compound of formula (II) in which W represents a hydrogen atom may be subjected to the action of a bromo derivative of formula (III):
   R2′-Br (III)
   in which R2′ has the meaning given above for R2 in which the optional reactive functions are optionally protected with protecting groups,
to give a product of formula (IV): 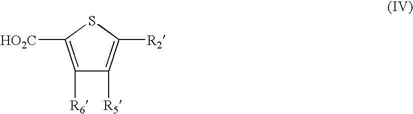
   in which R2′, R5′ and R6′ have the meanings given above,
when R2′ represents a —CH2-phenyl radical, to give the product of formula (IV1): 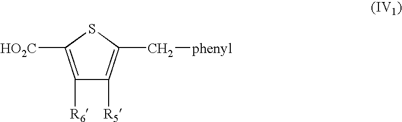
   in which R5′ and R6′ have the meanings given above, which product may be subjected to a second reaction with the compound of formula (III) to give the compound of formula (IV2): 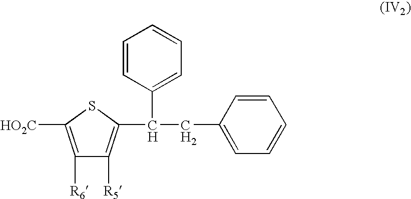
   in which R5′ and R6′ have the meanings given above, which products of formulae (IV), (IV1) and (IV2) may be subjected to an esterification reaction to give the corresponding products of formulae (V), (V1) and (V2): 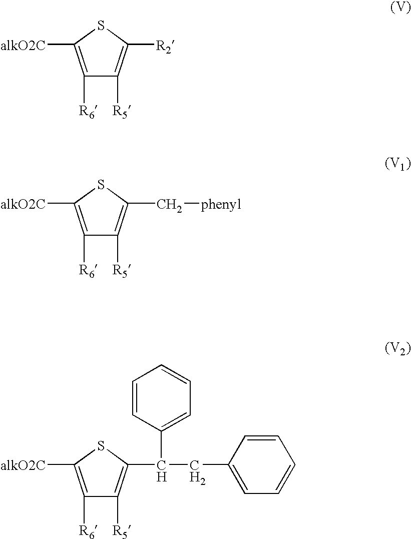
   in which R2′, R5′ and R6′ have the meanings given above and alk represents a linear or branched alkyl radical containing up to 4 carbon atoms,
route ii) the compound of formula (II) in which W represents a hydrogen atom may be subjected to an esterification reaction with an alcohol of formula (VI):
   Rc-OH (VI)
   in which Rc represents a linear or branched alkyl radical containing up to 6 carbon atoms,
to give products of formula (VII): 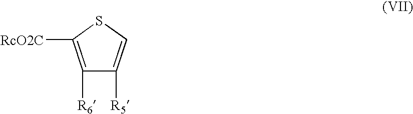
   in which Rc, R5′ and R6′ have the meanings given above, which products are subjected to an aldehyde of formula (VIII):
   Rd-CHO (VIII)
   in which Rd represents a hydrogen atom, an alkyl radical as defined above or a monocyclic radical or a radical consisting of saturated or unsaturated carbocyclic or heterocyclic fused rings that are up to 14-membered, containing one or more identical or different hetero atoms O, N, NH or S, which are themselves optionally substituted,
to give products of formula (IX): 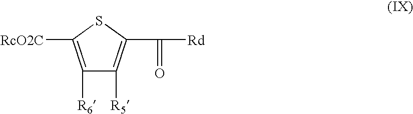
   in which Rc, Rd, R5′ and R6′ have the meanings given above,
which products of formula (IX) are reacted with an amine of formula (X)
   H2N—CH2-ORe (X)
   in which Re represents an optionally substituted arylalkyl radical,
to give products of formula (XI): 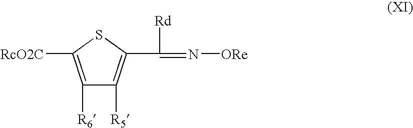
   in which Rc, Rd, Re, R5′ and R6′ have the meanings given above,
which product of formula (XI) may be subjected to a selective reduction reaction,
to give products of formula (XII): 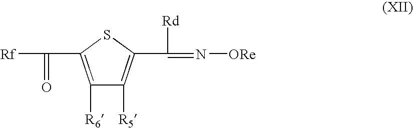
   in which Rd, Re, Rf, R5′ and R6′ have the meanings given above,
which products of formulae (XI) and (XII) may be subjected to a reduction reaction to give the corresponding products of formula (XIII): 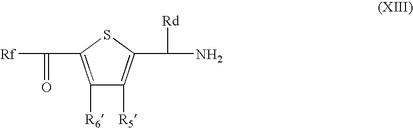
   in which Rd, Rf, R5′ and R6′ have the meanings given above,
which products of formula (XIII) may be subjected to a saponification reaction to give the corresponding products of formula (XIV): 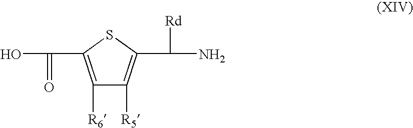
   in which Rd, R5′ and R6′ have the meanings given above, which product of formula (XIV) may be reacted with a product of formula (XV): 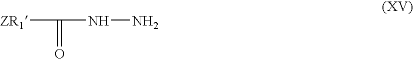
   in which R1′ has the meaning given above in which the optional reactive functions are optionally protected with protecting groups,
to give products of formula (I′x): 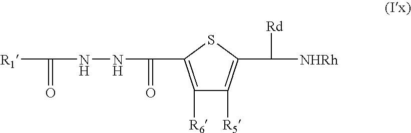
   in which R1′, Rd, R5′ and R6′ have the meanings given above and Rh represents —C(O)—O—R7 or —C(O)-alk-NH—C(O)—O—R7 with R7 as defined above,
b) when W represents a halogen atom, the product of formula (II) may be subjected to an esterification reaction with an alcohol of formula (VI) as defined above, to give products of formula (XVI): 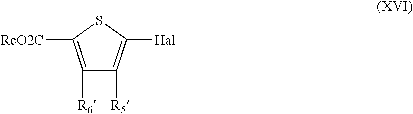
   in which Rc, R5′ and R6′ have the meanings given above and Hal represents a halogen atom,
which product of formula (XVI) may be subjected:
either to the action of a boron derivative of formula (XVII):
   R2′-B(OH)2 (XVII)
   in which R2′ has the meaning given above,
to give a product of formula (XVIII): 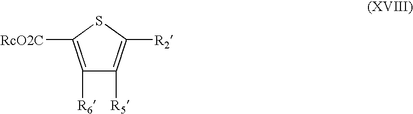
   in which Rc, R2′, R5′ and R6′ have the meanings given above,
or to the action of a product of formula (XIX):
   NH—RaRb (XIX)
   in which Ra and Rb, which may be identical or different, represent an optionally substituted alkyl or aryl radical as defined above,
to give a product of formula (XX): 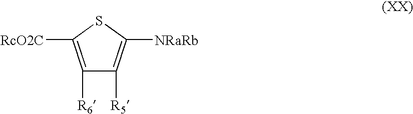
   in which Rc, Ra, Rb, R5′ and R6′ have the meanings given above,
c) when W represents a nitro radical, the product of formula (II) may be subjected to an esterification reaction with an alcohol of formula (VI) as defined above, to give products of formula (XXI): 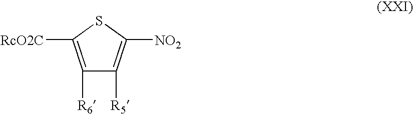
   in which Rc, R5′ and R6′ have the meanings given above, which product may be subjected to a reaction with a halo derivative of formula (XXII):
   RaRbHal (XXII)
   in which Ra and Rb have the meanings given above and Hal represents a halogen atom,
to give a product of formula (XX) as defined above,
which products of formulae (IV), (IV1), (IV2), (V), (VI), (V2), (scheme I) (XVIII) and (XX) as defined above may be reacted with a diamine NH2-NH2 to give a product of formula (XXIII): 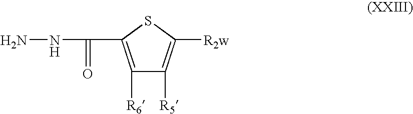
   in which R5′ and R6′ have the meanings given above and R2w represents —CH2-phenyl, —(CH)phenyl-CH2(phenyl), R2′ or NRaRb as defined above,
which product may be subjected to a reaction with a product of formula (XXIV):
   R1′-COOH (XXIV)
   in which R1′ has the meaning given above, to give a product of formula (Iy): 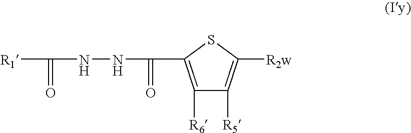
   in which R1′, R2w, R5′ and R6′ have the meanings given above,
which products of formulae (Ix′) and (Iy′) may be products of formula (I) and which, in order to obtain products or other products of formula (I), may be subjected, if desired and if necessary, to one or more of the following conversion reactions, in any order:
a) an esterification reaction of an acid function,
b) a saponification reaction of an ester function to an acid function,
c) an oxidation reaction of an alkylthio group to the corresponding sulfoxide or sulfone,
d) a reaction for conversion of a ketone function to an oxime function,
e) a reduction reaction of the free or esterified carboxyl function to an alcohol function,
f) a reaction for conversion of an alkoxy function to a hydroxyl function, or alternatively of a hydroxyl function to an alkoxy function,
g) an oxidation reaction of an alcohol function to an aldehyde, acid or ketone function,
h) a reaction for conversion of a nitrile radical to a tetrazolyl,
i) a reaction for removal of the protecting groups possibly borne by the protected reactive functions,
j) a salification reaction with a mineral or organic acid or with a base, to give the corresponding salt,
k) a reaction for resolving the racemic forms into resolved products,
said products of formula (I) thus obtained being in any possible racemic, enantiomeric or diastereoisomeric isomer form.

FIGURE 1 below represents the scheme for synthesizing the products of formula (I) according to the process defined above. Under preferred conditions for implementing the invention, the process described above may be performed in the following manner:

The product of formula (II) in which W represents a hydrogen atom may be subjected to the action of the halo derivative of formula (III) to give a product of formula (IV) in a solvent such as THF in the presence of LDA.

The product of formula (III) represents a halo derivative, for instance alkylHal or arylalkylHal in which Hal represents a halogen atom and aryl and alkyl respectively represent an aryl radical and an alkyl radical as defined above: (III) thus represents, for example, phenyl-CH2-Br or an alkyl bromide such as isopropyl bromide. The product of formula (IV) obtained may again be subjected to the action of the same compound of formula (III) under the same conditions.

Thus, when (III) represents phenyl-CH2-Br, the product of formula (IV1) obtained may again be subjected to the action of the same compound of formula (III) under the same conditions, and the product of formula (IV2) as defined above is thus obtained.

The products of formulae (IV1) and (IV2) are subjected to an esterification reaction under the usual conditions, for instance in an alcohol such as ethanol or methanol catalyzed with H₂SO₄ or by coupling with dicyclohexylcarbodiimide (DCC) or derivatives thereof or diazomethane in methylene chloride, to give the corresponding products of formulae (V), (V1) and (V2) as defined above.

The product of formula (II) in which W represents a hydrogen atom may be subjected to the action of an alcohol of formula (VI), for instance methanol or ethanol in a solvent such as SOCl₂ or under the conditions indicated above to give a product of formula (VII) as defined above. The product of formula (VII) thus obtained is reacted with an aldehyde of formula (VIII), for example in THF, to give a product of formula (IX) as defined above.

In the compound of formula (VIII) as defined above, Rd especially represents a hydrogen atom or an alkyl or aryl radical, optionally substituted, especially, with a hydroxyl, phenyl or hydroxyphenyl radical.

The reaction of the product of formula (IX) with the amine of formula (X) is performed in pyridine or in dichloromethane in the presence of triethylamine to give a product of formula (XI) as defined above.

The product of formula (XI) thus obtained is subjected to a coupling reaction in the presence of DCC or a derivative thereof in CH₂Cl₂ or DMF to give a product of formula (XII) as defined above.

The products of formulae (XI) and (XII) may be subjected to a reduction reaction in trifluoroacetic acid in the presence of Zn to give a product of formula (XIII) as defined above.

The product of formula (XIII) is subjected to a saponification reaction under the usual conditions, for example in a THF/water/base mixture, the base possibly being lithium hydroxide (LiOH) or sodium hydroxide, and the product of formula (XIV) is thus obtained.

The product of formula (XIV) is reacted with a product of formula (V) as defined above in CH₂Cl₂ in the presence of DMF with a coupling agent such as DCC or a derivative thereof, to give a product of formula (I′x) as defined above.

The product of formula (II) in which W represents a halogen atom may be subjected to an esterification reaction with an alcohol of formula (VI) as defined above, to give a product of formula (XVI), working, for example, under the same conditions as for the preparation of (VII) from (II) and (VI).

It may be noted that the halogen atom is preferably a bromine atom.

The product of formula (XVI) thus obtained may be reacted with a boron derivative of formula (XVII), for example in toluene in the presence of Pd(P(phenyl)₃)₄ to give a product of formula (XVIII) as defined above.

The product of formula (XVI) thus obtained may also be reacted with an amine of formula (XIX) as defined above in DMF, preferably under pressure at 165° C., to give a product of formula (XX) as defined above.

The product of formula (II) in which W represents a nitro radical may be subjected to an esterification reaction with an alcohol of formula (VI) as defined above, to give a product of formula (XXI), working, for example, under the same conditions as for the preparation of (VII) from (II) and (VI).

The product of formula (XXI) thus obtained may be reacted with a product of formula (XXII) as defined above in DMF, to give the product of formula (XX) as defined above.

The products of formulae (IV), (IV1), (IV2), (V), (V1), (V2), (XVIII) or (XX) may then be reacted with hydrazine in hydrate form in a solvent such as, for example, DMF in an alcohol such as, for example, ethanol, to give the corresponding products of formula (XXIII) as defined above.

The products of formula (XXIII) thus obtained may then be reacted with an acid of formula (XXIV), for example in dichloromethane and DMF, to give a product of formula (Iy′) as defined above.

The product of formula (XXIV) R1′-COOH may especially represent an amino acid residue, for instance Phe or Leu, which is optionally substituted.

Depending on the values of R1′, R5′, R6′, Rd, Rh and R2W, the products of formulae (Ix′) and (Iy′) thus obtained may or may not constitute products of formula (I).

The products of formulae (Ix′) and (Iy′) may constitute products of formula (I) in which the functions that may be reactive are protected, and which thus give, after deprotection of these functions, products of formula (I) as defined above.

The products of formulae (Ix′) and (Iy′) may also give products of formula (I), or may be converted into other products of formula (I) by being subjected to one or more of the reactions a) to k) indicated above and detailed below.

Thus, the various reactive functions that may be borne by some of the compounds of the reactions defined above may, if necessary, be protected: they are, for example, hydroxyl, acyl, free carboxyl or amino and monoalkylamino radicals, which may be protected with suitable protecting groups.

The following nonexhaustive list of examples of protection of reactive functions may be mentioned:
the hydroxyl groups may be protected, for example, with alkyl radicals such as tert-butyl, trimethylsilyl, tert-butyldimethylsilyl, methoxymethyl, tetrahydropyranyl, benzyl or acetyl,
the amino groups may be protected, for example, with acetyl, trityl, benzyl, tert-butoxycarbonyl, benzyloxycarbonyl or phthalimido radicals or with other radicals known in peptide chemistry,
the acyl groups, such as the formyl group, may be protected, for example, in the form of ketals or cyclic or noncyclic thioketals such as dimethyl or diethyl ketal or ethylenedioxy ketal, or diethylthio ketal or ethylenedithio ketal,
the acid functions in the products described above may be, if desired, amidated with a primary or secondary amine, for example in methylene chloride in the presence, for example, of 1-ethyl-3-(dimethylamino-propyl)carbodiimide hydrochloride at room temperature,
the acid functions may be protected, for example, in the form of esters formed with readily cleavable esters, such as benzyl or tert-butyl esters, or esters known in peptide chemistry.

The reactions to which the products of formulae (IV) and (I′) as defined above may be subjected, if desired or if necessary, may be performed, for example, as indicated below.

a) The products described above may, if desired, undergo, on the possible carboxyl functions, esterification reactions that may be performed according to the usual methods known to those skilled in the art.

b) The possible conversions of ester functions to acid functions of the products described above may be, if desired, performed under the usual conditions known to those skilled in the art, especially by acid or alkaline hydrolysis, for example with sodium hydroxide or potassium hydroxide in alcoholic medium, for instance in methanol, or alternatively with hydrochloric acid or sulfuric acid.

c) The possible alkylthio groups in the products described above may be, if desired, converted into the corresponding sulfoxide or sulfone functions under the usual conditions known to those skilled in the art, for instance with peracids, for instance peracetic acid or meta-chloroperbenzoic acid, or alternatively with ozone, oxone or sodium periodate, in a solvent, for instance methylene chloride or dioxane, at room temperature.

The production of the sulfoxide function may be promoted by means of an equimolar mixture of the product containing an alkylthio group and of the reagent such as, especially, a peracid.

The production of the sulfone function may be favored by means of a mixture of the product containing an alkylthio group with an excess of the reagent such as, especially, a peracid.

d) The reaction for conversion of a ketone function into an oxime may be performed under the usual conditions known to those skilled in the art, such as, especially, an action in the presence of an optionally O-substituted hydroxylamine in an alcohol, for instance ethanol, at room temperature or with heating.

e) The possible free or esterified carboxyl functions in the products described above may be, if desired, reduced to alcohol functions via the methods known to those skilled in the art: the possible esterified carboxyl functions may be, if desired, reduced to alcohol functions via the methods known to those skilled in the art, and especially with lithium aluminum hydride in a solvent, for instance tetrahydrofuran, dioxane or ethyl ether. The possible free carboxyl functions in the products described above may be, if desired, reduced to alcohol functions especially using boron hydride.

f) The possible alkoxy functions such as, especially, methoxy, in the products described above may be, if desired, converted into hydroxyl functions under the usual conditions known to those skilled in the art and especially under the conditions described below in the experimental section.

g) The possible alcohol functions in the products described above may be, if desired, converted into aldehyde or acid functions by oxidation under the usual conditions known to those skilled in the art, for instance via the action of manganese oxide to give aldehydes, or of Jones' reagent to give acids.

h) The possible nitrile functions in the products described above may be, if desired, converted into tetrazolyl under the usual conditions known to those skilled in the art, for instance by cycloaddition of a metal azide, for instance sodium azide or a trialkyltin azide on the nitrile function, as indicated in the method described in the article referenced as follows: J. Organometallic Chemistry, 33, 337 (1971) Kozima S. et al.

It may be noted that the reaction for conversion of a carbamate into a urea and especially of a sulfonylcarbamate into a sulfonylurea may be performed, for example, at the reflux temperature of a solvent, for instance toluene, in the presence of the appropriate amine. It is understood that the reactions described above may be performed as indicated or, where appropriate, according to other usual methods known to those skilled in the art.

i) The removal of the protecting groups, for instance those indicated above, may be performed under the usual conditions known to those skilled in the art, especially via an acid hydrolysis performed with an acid such as hydrochloric acid, benzenesulfonic acid or para-toluenesulfonic acid, formic acid or trifluoroacetic acid, or alternatively by catalytic hydrogenation.

The phthalimido group may be removed with hydrazine. A list of the various protecting groups that may be used will be found, for example, in patent BF 2 499 995.

j) The products described above may, if desired, undergo salification reactions, for example with a mineral or organic acid or with a mineral or organic base according to the usual methods known to those skilled in the art.

k) The possible optically active forms of the products described above may be prepared by resolving the racemic mixtures according to the usual methods known to those skilled in the art.

Illustrations of such reactions defined above are given in the preparation of the examples described below.

The products of formula (I) as defined above and also the addition salts thereof with acids have advantageous pharmacological properties.

The products of the present invention may thus be endowed with inhibitory properties on one or more metabolic enzymes as defined above, and especially on certain protein kinases or proteases.

Several kinase inhibitors have been described, for instance butyrolactone, flavopiridol and 2-(2-hydroxyethylamino)-6-benzylamino-9-methylpurine, also known as olomucine.

Several protease inhibitors have also been described, especially in “Journal of Medicinal Chemistry”, Vol. 43 No 3 (D. Leung, G. Abbenante and D. P. Fairlie). A nonexhaustive list of thrombin inhibitors that may be mentioned, for example, includes: argatroloan, napsagatran, inogatran, efegatran, CVS-1123 and melagatran.

Powerful factor Xₐ inhibitors that may also be mentioned include DX-9065ₐ, YM-60828 and ZK 807-191 or FX 2212.

Elastase inhibitors that may be mentioned include ICI-200800, MR 889, L-658,758, MDL 101,146 and ZD 8321, or alternatively various heterocyclic derivatives such as penicillins, penems, β-lactams, isocoumarins, benzisothiazolones and alkylazetidinones.

A tryptase inhibitor that may be mentioned is APC-366, and “complement convertase” inhibitors that may be mentioned include sepimostat mesylate (FUT-187) and nafamostat mesylate (FUT 175).

Powerful cathepsin K inhibitors that may also be mentioned include Cbz-Leu-Leu-Leu-CHO, 1,3-bis(acylamino)-2-propanone or 1,5-diacylcarbohydrazide derivatives; caspase inhibitors that may be mentioned include 5-aminopyrimidin-6-one derivatives and phenyl ketomethyl ether or aminomethylene ketone derivatives; calpain inhibitors that may be mentioned include peptide aldehydes such as Cbz-Val-Phe-CHO and calpeptin.

The products of the present invention as defined above have inhibitory properties on kinases or proteases and especially on cysteine proteases. The products of the present invention as defined above thus especially have inhibitory properties on cathepsins B, H, J, L, N, S, T, C, V, W, K or O, O2 and most particularly on cathepsin K.

The products of the present invention or the pharmaceutically acceptable salts thereof are thus especially useful for preventing or treating diseases requiring the use of kinase or protease inhibitors, especially cysteine protease inhibitors, particularly cathepsin B, H, J, L, N, S, T, C, V, W, K or O, O2 inhibitors and most particularly cathepsin K inhibitors.

The levels, the regulation and the activity of a certain number of protein kinases or proteases play a role in several human pathologies. The activity of a protein kinase may especially be associated with receptors having transmembrane domains or with intracellular proteins.

Certain kinases or proteases may play a role in the initiation, development and completion of cell cycle events, and thus molecules inhibiting such kinases or proteases are liable to limit undesired cell proliferations such as those observed in cancer, psoriasis and the growth of fungi or parasites (animals or protists): such molecules inhibiting these kinases or proteases are thus also capable of intervening in the regulation of neurodegenerative diseases such as Alzheimer's disease.

Certain products of formula (I) of the present invention may thus be endowed with antimitotic properties.

Certain products of formula (I) as defined above may, like kinase or protease inhibitors, especially have the property of inhibiting osteoclast-mediated bone resorption. They may thus be useful for the therapeutic or prophylactic treatment of diseases that are at least partially caused by an undesired increase in bone resorption, for example osteoporosis. Certain products of formula (I) of the present invention may thus, for example, inhibit the adhesion of osteoclasts to the surface of bone and thus bone resorption by the osteoclasts.

The bone diseases whose treatment or prevention requires the use of compounds of formula (I) or prodrugs thereof are especially osteoporosis, hypercalcemia, osteopenia, for example caused by bone metastases, dental disorders, for example periodontitis, hyperparathyroidism, periarticular erosions in rheumatoid arthritis, Paget's disease, and immobilization-induced osteopenia. In addition, the compounds of formula (I) may be used to alleviate, prevent or treat bone disorders that are caused by treatments, glucocorticoids therapies associated with the taking of steroids or corticosteroids, or by deficiencies in male or female sexual hormones.

All these disorders are characterized by bone loss, which is based on a defect of equilibrium between bone formation and bone destruction and which may be favorably influenced by inhibiting bone resorption by the osteoclasts.

Certain products of formula (I) of the present invention may have, in addition to their specific inhibitor properties on kinases or proteases, advantageous cellular effects such as antiproliferative properties and especially effects on apoptosis.

It is known from studies described in the literature, such as in WO 97/20842, that relationships exist between the cell cycle and apoptosis. Among the routes leading to apoptosis, some are dependent on kinases or proteases.

The products of the present invention are especially useful for tumor therapy.

The products of the invention may thus also enhance the therapeutic effects of commonly used antitumor agents.

The products of formula (I) of the present invention thus most particularly have antimitotic and anti-neurodegenerative properties.

Certain products of the present invention may be inhibitors of vasoconstrictive and hypertensive effects and may thus produce an antiischemic effect, or alternatively may oppose stimulatory effects on certain cell types, especially smooth muscle cells, fibroblasts, neuronal cells and bone cells.

The products according to the present invention may thus be used in the treatment of diseases such as proliferative diseases, cancer, restenosis or inflammation; allergies, cardiovascular diseases or certain infectious diseases.

The products of the present invention may also be used in the treatment of certain gastrointestinal or gynecological disorders and in particular for a relaxing effect on the uterus.

These properties justify their therapeutic application and a subject of the invention is particularly, as medicinal products, the products of formula (I) as defined above, said products of formula (I) being in any possible racemic, enantiomeric or diastereoisomeric isomer form, and also the pharmaceutically acceptable salts with mineral and organic acids or with mineral and organic bases of said products of formula (I).

A subject of the invention is thus particularly, as medicinal products, the products of formula (I) as defined above, and also the pharmaceutically acceptable addition salts with mineral and organic acids or with mineral and organic bases of said products of formula (I).

A subject of the invention is more particularly, as medicinal products, the products of formula (Ia) or (Ib) as defined above, and also the pharmaceutically acceptable addition salts with mineral and organic acids or with mineral and organic bases of said products of formula (Ia) or (Ib).

A subject of the invention is most particularly, as medicinal products, the products described below in the examples and especially the products of formula (I) as defined above, corresponding to the following formulae:
5-[(2S)-1-[[4-methyl-1-oxo-2-[[(phenylmethoxy)-carbonyl]amino]pentyl]amino]ethyl]-2-thiophenecarboxylic acid 2-[(2S)-4-methyl-1-oxo-2-[[(phenylmethoxy)carbonyl]amino]pentyl]hydrazide
5-[1-(hydroxyimino)ethyl]-2-thiophenecarboxylic acid 2-[[(9H-fluoren-9-yl)methoxy]carbonyl]hydrazide
5-[2-(phenylmethoxy)phenyl]-2-thiophenecarboxylic acid 2-[(2S)-4-methyl-1-oxo-2-[[(phenylmethoxy)-carbonyl]amino]pentyl]hydrazide
5-[[[3-(4-methoxyphenoxy)phenyl]methyl](phenylmethyl)amino]-2-thiophenecarboxylic acid 2-[(2S)-4-methyl-1-oxo-2-[[(phenylmethoxy)carbonyl]amino]pentyl]-hydrazide
5-[1-[(phenylmethoxy)imino]ethyl]-2-thiophenecarboxylic acid 2-[(2S)-4-methyl-1-oxo-2-[[(phenylmethoxy)carbonyl]amino]pentyl]hydrazide
5-[1-[[(phenylmethoxy)carbonyl]amino]ethyl]-2-thiophenecarboxylic acid 2-[[(9H-fluoren-9-yl)methoxy]-carbonyl]hydrazide
5-[1-[[(phenylmethoxy)carbonyl]amino]ethyl]-2-thiophenecarboxylic acid 2-[(2S)-4-methyl-1-oxo-2-[[(phenylmethoxy)carbonyl]amino]pentyl]hydrazide
5-[3-methyl-1-[(phenylmethoxy)carbonyl]amino]butyl]-2-thiophenecarboxylic acid 2-[(2S)-4-methyl-1-oxo-2-[[(phenylmethoxy)carbonyl]amino]pentyl]hydrazide
5-[3-methyl-1-[[(phenylmethoxy)carbonyl]amino]butyl]-2-thiophenecarboxylic acid 2-[(2S)-4-methyl-1-oxo-2-[[(phenylmethoxy)carbonyl]amino]pentyl]hydrazide isomer A
5-[3-methyl-1-[[(phenylmethoxy)carbonyl]amino]butyl]-2-thiophenecarboxylic acid [(2S)-4-methyl-1-oxo-2-[[(phenylmethoxy)carbonyl]amino]pentyl]hydrazide isomer B
and also the pharmaceutically acceptable addition salts with mineral and organic acids or with mineral and organic bases of said products of formula (I).

The products of formula (I) of the present invention may thus especially be used in the form of medicinal products for inhibiting an undesired mechanism causing a pathology due to the effect of one or more protease(s) or kinase(s): the method may thus consist in administering to the patient whose treatment requires the inhibition of the effect of such a protease or kinase, an inhibitory amount of such a medicinal product according to the present invention.

The medicinal products that are the subject of the invention may thus be used in the treatment and prevention of cardiovascular complaints associated with an impairment in vasomotor action or bulimia, myocardial infarction and its consequences, heart failure, renal failure, angina pectoris, hyperaldosteronism, arterial hypertension and its consequences, the treatment and prevention of complications, in particular cardiac and vascular hypertrophies and also the development of fibroses in target organs, the prevention and treatment of hypertension, metabolic, endocrinological and neurological disorders, endotoxic shock, “subarachnoid” hemorrhaging, arrhythmia, asthma, acute renal failure, preeclampsia and diabetes.

The medicinal products that are the subject of the invention may thus be used in the treatment of vascular spasms, in the treatment of the sequels to a cerebral hemorrhage, or in the treatment of coronary spasms or peripheral vascular spasms. These medicinal products may especially be used in the treatment of congestive heart failure, in the prevention of post-angioplasty restenosis, in the prevention of cardiac and vascular fibroses, in the treatment of atherosclerosis and certain forms of hypertension, for instance pulmonary hypertension, and also in the treatment of asthma.

These medicinal products forming the subject of the invention may also be used for treating glaucoma and various types of visceral spasms, and also as neuronal protective substances, or in the prevention of post-angioplasty restenosis.

The medicinal products that are the subject of the invention may especially be used for their cardiac and vascular antihypertrophic and antifibrotic effects. Most particularly, they may be used for treating and preventing diabetes-related cardiovascular disorders.

The medicinal products that are the subject of the present invention may also find an application in the treatment of osteoporosis and as neuronal protectors.

The medicinal products that are the subject of the invention may also be used in the treatment of memory and cognitive function disorders and also anxiety.

The medicinal products that are the subject of the invention may also be used, like antimitotic agents, in cancer chemotherapy, or alternatively in the treatment of psoriasis, parasitoses such as those caused by protists or fungi, or alternatively in the treatment of Alzheimer's disease or in the treatment of neuronal apoptosis.

The invention covers pharmaceutical compositions containing as active principle at least one of the medicinal products as defined above.

Such pharmaceutical compositions of the present invention may also, where appropriate, contain active principles of other antimitotic medicinal products such as, especially, those based on taxol, cis-platin, DNA intercalating agents and the like.

These pharmaceutical compositions may be administered orally, parenterally or locally by topical application to the skin and mucous membranes, or by intravenous or intramuscular injection.

These compositions may be solid or liquid and may be in any pharmaceutical form commonly used in human medicine, for instance simple or sugar-coated tablets, pills, lozenges, gel capsules, drops, granules, injectable preparations, ointments, creams or gels; they may be prepared according to the usual methods. The active principle may be incorporated therein with excipients usually used in these pharmaceutical compositions, such as talc, gum arabic, lactose, starch, magnesium stearate, cocoa butter, aqueous or nonaqueous vehicles, fatty substances of animal or plant origin, paraffin derivatives, glycols, various wetting, dispersing or emulsifying agents, and preserving agents.

The usual dosage, which varies according to the product used, the individual treated and the complaint under consideration, may be, for example, from 0.05 to 5 g per day, and preferably from 0.1 to 2 g per day, in adults.

A subject of the invention is most particularly the pharmaceutical compositions as defined above, characterized in that they contain one or more product(s) of formula (I) as defined above: such pharmaceutical compositions are thus characterized in that they contain one or more kinase or protease inhibitors as defined above and in that they are used as medicinal products for the therapeutic applications indicated above.

A subject of the invention is particularly the use of the products of formula (I) as defined above for the preparation of pharmaceutical compositions for treating or preventing complaints requiring an inhibition of one or more metabolic enzymes as defined above.

A subject of the invention is thus particularly the use of the products of formula (I) as defined above for the preparation of pharmaceutical compositions for treating diseases resulting from anomalies in the levels, regulation or activity of a certain number of metabolic enzymes. Such anomalies play a role in certain human pathologies with which the present invention is particularly concerned.

The present invention thus especially relates to the use of the products of formula (I) of the present invention as protease or kinase inhibitors. The present invention thus relates to the inhibition of certain of such protease or kinase enzymes, and claims the use of products of the present invention as inhibitors in cases in which the inhibition of such a protease or kinase is indicated.

A subject of the invention is thus most particularly the use of the products of formula (I) as defined above or of pharmaceutically acceptable salts of said products of formula (I) for the preparation of medicinal products for preventing or treating medicinal products for preventing or treating diseases in which metabolic enzymes such as proteases or kinases are involved.

A subject of the present invention is thus the use of products of formula (I) as defined above or of pharmaceutically acceptable salts of said products of formula (I) for the preparation of medicinal products for preventing or treating diseases associated with an abnormal physiological behavior in the secretion and/or the activity of cysteine proteases such as, especially, the cathepsins B, H, J, L, N, S, T, C, V, W, K or O, O2 or papain.

A subject of the present invention is, more specifically, the use of products of formula (I) as defined above or of pharmaceutically acceptable salts of said products of formula (I) for the preparation of medicinal products for preventing or treating diseases associated with an abnormal physiological behavior in the secretion and/or the activity of cathepsin K.

A subject of the present invention is especially the use of products of formula (I) as defined above or of pharmaceutically acceptable salts of said products of formula (I) as inhibitors of the cysteine protease cathepsin K.

A subject of the present invention is thus especially the use of products of formula (I) as defined above or of pharmaceutically acceptable salts of said products of formula (I) for the preparation of medicinal products for preventing or treating diseases requiring the use of inhibitors of the cysteine protease cathepsin K.

A subject of the present invention is particularly the use of products of formula (I) as defined above or of pharmaceutically acceptable salts of said products of formula (I) for the preparation of medicinal products for preventing or treating cartilage and bone metabolism disorders, proliferative diseases, cancers, cardiovascular diseases, restenosis, central nervous system diseases, immune system diseases, allergies, infectious and inflammatory diseases and autoimmune diseases.

A subject of the invention is thus, most particularly, the use of the products of formula (I) as defined above for the preparation of pharmaceutical compositions for treating arterial hypertension, heart failure, post-angioplasty restenosis, vascular spasms, the sequels to a cerebral hemorrhage and renal failure.

A subject of the invention is thus, most particularly, the use of the products of formula (I) as defined above for the preparation of pharmaceutical compositions for treating myocardial infarction, for preventing post-angioplasty restenosis and for preventing cardiac and vascular fibroses.

A subject of the invention is thus, most particularly, the use of the products of formula (I) as defined above for the preparation of pharmaceutical compositions for treating kidney failure.

A subject of the invention is thus, most particularly, the use of the products of formula (I) as defined above for the preparation of pharmaceutical compositions for treating and preventing diabetes-related cardiovascular disorders.

A subject of the invention is especially the use of the products of formula (I) as defined above for the preparation of medicinal products for cancer chemotherapy, for treating psoriasis, parasitoses such as those caused by fungi or protists, for treating Alzheimer's disease or for treating neurodegenerative diseases, especially neuronal apoptosis.

A subject of the present invention is, more particularly, the use as defined above, characterized in that the diseases to be prevented or treated are chosen from cartilage and bone metabolism diseases, proliferative diseases, cancers and neurodegenerative diseases, for instance Alzheimer's disease.

A subject of the present invention is most particularly the use as defined above, characterized in that the diseases to be prevented or treated are chosen from cartilage and bone metabolism diseases and bone cancer.

A subject of the present invention is especially the use as defined above, characterized in that the diseases to be prevented or treated are especially osteoporosis, gingival diseases including gingivitis and periodontosis, arthritis such as, especially, osteoarthritis and rheumatoid arthritis, Paget's disease, hypercalcemia and bone cancer.

A subject of the present invention is thus the use as defined above, characterized in that the disease to be prevented or treated is especially osteoporosis.

The preparation of the products of formula (I) as defined above involves starting compounds described above of various formulae, such as the compounds of formula (II) in which W represents a hydrogen atom, a halogen atom or a nitro radical, the alcohols of formulae (III) and (VI), the aldehydes of formula (VIII), amines of formula (X), diamines of formula (XV) (R1′-CO—NR3-NR4-), boron derivatives of formula (XVII) (R2′-B—(OH)2), diamines of formula (XIX), reagents of formula (XXII) and acids of formula (XXIV) (R1′-COOH).

Among such starting materials, some are known and may be commercially obtained. Certain starting materials may be prepared according to the usual methods known to those skilled in the art.

It is also especially possible to prepare certain starting materials from commercial products, for example by subjecting them to one or more of the reactions described above in a) to k), performed under the conditions that are also described above.

The starting materials of formula (XXIV) may be natural or unnatural amino acids, commercially available or prepared according to the usual methods known to those skilled in the art.

Mention may be made especially of the following amino acids: Glu, Gly, Ala, Val, Phe or Tyr, which are optionally in a protected form, for example protected with an Fmoc, alloc or tert-butyl group.

It is especially possible, starting with “natural” or “unnatural” amino acids, to attach an Fmoc radical to the N-terminal amine function of the amino acid under consideration: such a reaction is especially described in the article whose reference follows: Mueller, A.; Vogt, C.; Sewald, N.; Synthesis (1998), (6), 837-841.

A large number of amino acids in fmoc protected form are also found commercially, and may thus constitute starting materials for the process of the present patent application. Mention may be made especially of Fmoc-Leu, Fmoc-L-Glu (Alloc); Fmoc-Gly; Fmoc-Ala; Fmoc-Val; Fmoc-Phe or Fmoc-Tyr(OtBu).

The experimental section below gives examples of such starting materials.

Finally, a subject of the present invention is, as novel industrial products, the products of formulae (XIII), (XIV) and (XXIII) as defined above.

The examples that follow illustrate the invention without, however, limiting it.

### BRIEF DESCRIPTION OF THE DRAWING

FIGURE 1 shows the general scheme for synthesizing compounds of formula (I).

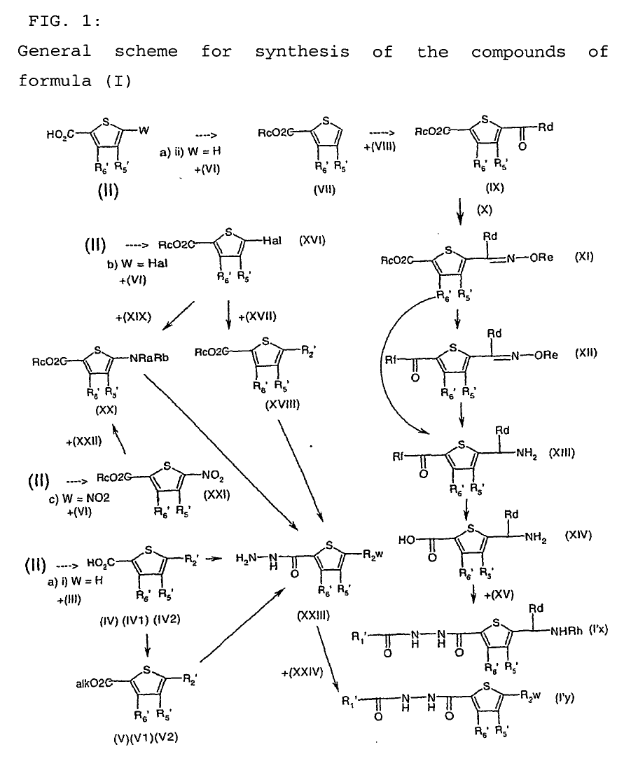

### EXPERIMENTAL SECTION

### Example 1

### 5-[(2S)-1-[[4-Methyl-1-oxo-2-[[(phenylmethoxy)carbonyl]amino]pentyl]amino]ethyl-2-thiophenecarboxylic Acid 2-[(2S)-4-methyl-1-oxo-2-[[(phenylmethoxy)carbonyl]amino]pentyl]hydrazide

### Stage 1: 5-[1-(hydroxyimino)ethyl]-2-thiophenecarboxylic Acid

1.84 g of hydroxylamine hydrochloride are added to a solution of 15 ml of pyridine under argon, containing 2.25 g of 5-acetyl-2-thiophenecarboxylic acid. The medium is maintained at 100° C. for 8 hours and then concentrated to dryness, taken up in 20 ml of 2N HCl solution, extracted with 50 ml of ethyl acetate, dried over MgSO₄, filtered and evaporated under vacuum. The residue is purified on silica, eluting with 95/5/0.5 CH₂Cl₂/MeOH/CH₃COOH, and 0.450 g of the expected product is thus obtained.

NMR (1H, DMSO): 2.17 (s, 3H); 2.28 (s, 3H); 7.37 (d, 1H); 7.49 (d, 1H); 7.55 (d, 1H); 7.70 (d, 1H); 11.57 (s, 1H); 12.13 (broad s, 1H); 13.08 (broad s, 1H).

### Stage 2: 5-[1-(Hydroxyimino)ethyl]-2-thiophenecarboxylic Acid [(2,2-dimethylethoxy)carbonyl]hydrazide

0.28 g of HOBT (1-hydroxybenzotriazole hydrate) is added at about 0-5° C. (ice-salt bath) to a solution of 2 ml of DMF and 3 ml of CH₂Cl₂ containing the product obtained in stage 1 above, followed by addition of 0.39 g of EDC 1-(3-dimethylaminopropyl)-3-ethylcarbodiimide hydrochloride). The reaction medium is brought to room temperature and stirred for 30 minutes. Boc-hydrazine dissolved in 1 ml of CH₂Cl₂ is added at about 0-5° C. The medium is stirred for 3 hours at room temperature.

After evaporation under vacuum, the residue is taken up in 50 ml of ethyl acetate and washed with 25 ml of an aqueous solution (pH=4). The ethyl acetate solution is separated out by settling of the phases, and then washed with 20 ml of an aqueous solution. The organic phase is then separated out, dried and evaporated under vacuum. The residue is purified by chromatography on silica, eluting with a 90/10 dichloromethane/methanol solution. 0.460 g of the expected product is thus obtained.

NMR (1H, DMSO) 1.47 (s, 9H); 2.17 (s, 3H); 2.27 (s, 3H); 7.37 (d, 1H); 7.50 (d, 1H); 7.71 (d, 1H); 7.77 (d, 1H); 8.95 (broad s, 1H); 10.24 (broad s, 1H); 10.27 (s, 1H); 11.51 (s, 1H); 12.0 (s, 1H).

### Stage 3: 5-(1-aminoethyl)-2-thiophenecarboxylic Acid Hydrazide bis(trifluoroacetate)

Reduction of the oxime and deprotection of the carbamate are performed as follows.

0.44 g of Zn is added to a solution under argon at room temperature containing 0.2 g of the oxime derivative obtained in stage 2 above. The suspension obtained is stored for 1 hour and then filtered through Celite. The filtered solution is concentrated and then taken up in 50 ml of ethyl ether. The precipitate obtained is filtered off. 0.20 g of the expected product is thus obtained.

### Stage 4: 5-[(2S)-1-[[4-methyl-1-oxo-2-[[(phenylmethoxy)carbonyl]amino]pentyl]amino]ethyl]-2-thiophenecarboxylic Acid 2-[(2S)-4-methyl-1-oxo-2-[[(phenylmethoxy)carbonyl]amino]pentyl]hydrazide

0.18 g of HOBT is added to a solution of 2 ml of DMF and 2 ml of CH₂Cl₂ containing 0.35 g of a derivative Z-Leu, followed by addition of 0.26 g of EDC at 0° C. The mixture is then brought to room temperature over 30 minutes and 0.24 g of thiophene derivative, obtained in stage 3 above, dissolved in 2 ml of DMF is added. This mixture is maintained at room temperature for 12 hours and then evaporated under vacuum, taken up in 50 ml of ethyl acetate and washed with 25 ml of 2N HCl solution and then with 25 ml of saturated NaCl solution. The resulting solution is dried over MgSO₄, filtered and purified by chromatography, eluting with a 95/5 dichloromethane/methanol solution. 0.070 g of the expected product is thus recovered.

NMR (1H, DMSO) 0.86 (m, 12H); 1.32 to 1.78 (m, 6H); 4.06 (m, 1H); 4.16 (m, 1H); 5.03 (s, 4H); 5.14 (m, 1H); 6.96 (d, 1H); 7.0 (d, 1H); 7.35 (m, 10H); 7.66 (d, 1H); 7.67 (d, 1H); 7.39 (d, 1H); 7.52 (d, 1H); 8.52 (d, 1H); 8.56 (d, 1H); 10.04 (broad s, 1H); 10.32 (broad s, 1H).

### Example 2

### 5-[1-(Hydroxyimino)ethyl]-2-thiophenecarboxylic Acid 2-[[(9H-fluoren-9-yl)methoxy]carbonyl]-hydrazide

0.53 g of HOBT is added to a solution of 5 ml of CH₂Cl₂ and 3 ml of DMF at 0° C. (ice-salt bath) containing 0.73 g of the product obtained in stage 1 of Example 1, followed by addition of 0.75 g of EDC. The mixture is warmed to room temperature and maintained for 20 minutes. 1 g of Fmoc-hydrazine dissolved in 5 ml of DMF are added to this solution at 0-5° C. (ice-salt bath). The medium is then warmed to room temperature and maintained at that temperature for 6 hours, and then evaporated to dryness and taken up in 50 ml of ethyl acetate and 25 ml of H₂O. The ethyl acetate solution is separated out by settling of the phases, dried over MgSO₄, concentrated and purified by chromatography on silica, eluting with a 98/2/0.5 CH₂Cl₂/MeOH/CH₃COOH solution to give 1.42 g of the expected product.

NMR (1H, DMSO): 2.17 (s, 3H); 2.28 (s, 3H); 4.23 (m, 3H); 4.28 (broad s, 1H); 4.40 (broad d, 1H); 7.05 to 7.70 (m, 2H); 7.35 (broad t, 2H); 7.45 (broad t, 2H); 7.75 (broad d, 2H); 7.91 (broad d, 2H); 9.09 (s, 1H); 9.44 (s, 1H); 10.39 (s, 1H); 10.62 (s, 1H); 11.52 (s, 1H); 12.04 (s, 1H).

### Example 3

### 5-(4-Methoxyphenyl)-2-thiophenecarboxylic Acid 2-[(2S)-1-oxo-2-[[(phenylmethoxy)carbonyl]amino]-pentyl]hydrazide

### Stage 1: Ethyl 5-bromo-2-thiophenecarboxylate

10 g of 5-bromo-2-thiophenecarboxylic acid (48.29 mM, 1 equivalent) are stirred in 17.6 ml of SOCl₂ (24 mM, 5 equivalents) for 1 hour 30 minutes at 100° C. The mixture is evaporated to dryness and taken up with twice 20 ml of toluene. The crude product obtained is stirred for 2 hours at reflux in 40 ml of EtOH. The mixture is evaporated to dryness, the residue is taken up in CH₂Cl₂ and washed with water, and then dried over MgSO₄ and evaporated to dryness. 10.95 g (96%) of the expected product are thus obtained in the form of a brown liquid.

### Stage 2: Ethyl 5-(4-methoxyphenyl)-2-thiophenecarboxylate

2.58 g (17.01 mM, 2 equivalents) of boronic acid and 18 ml of 2M sodium carbonate are added to a solution of 2.0 g of the bromo ester (8.5 mM, 1 equivalent) obtained in stage 1 above in 48 ml of toluene and 74 ml of EtOH, and the resulting mixture is degassed for 5 minutes with N₂. 1.21 g of Pd(PØ₃)₄ (1.05 mM, 0.124 equivalent) are then added. The mixture is stirred for 3 hours at 80° C. (external temperature). 200 ml of H₂O are added and the resulting mixture is extracted with 100 ml of EtOAc, dried over MgSO₄ and evaporated to dryness. 5.28 g of crude product are thus obtained, and are purified on a column of silica, eluting with 98/2 and then 95/5 cyclohexane/EtOAc. 2.22 g of expected product are thus obtained.

### Stage 3: 5-(4-methoxyphenyl)-2-thiophenecarboxylic Acid Hydrazide

200 ml of the ester (0.762 mM, 1 equivalent) obtained in stage 2 above are stirred at reflux in 5 ml of DMF and 1.1 ml of hydrazine hydrate (22.8 mM, 30 eq.) for 24 hours and then evaporated to dryness.

After purification on a column of silica, eluting with 5/5 CH₂Cl₂/EtOAc and then 9/1/0.1 CH₂Cl₂/MeOH/NH₄OH, 245 mg of expected product are obtained.

### Stage 4: 5-(4-methoxyphenyl)-2-thiophenecarboxylic Acid 2-[(2S)-1-oxo-2-[[(phenylmethoxy)carbonyl]amino]-pentyl]hydrazide

70 mg of keto hydrazide (0.282 mM, 1 equivalent) obtained in stage 3 above, 70 mg of EDC (0.366 mM, 1.3 equivalents) and 49 mg of HOBT (0.366 mM, 1.3 equivalents) are added to a solution of 97.0 mg of ZLeu-OH (0.366 mM, 1.3 equivalents) in 5 ml of DMF on siliporite and cooled to 0° C.

The mixture is stirred for 2 hours at 0° C. and for 2 hours at room temperature, the DMF is then evaporated off and the residue is taken up in 20 ml of Et₂O, washed with 20 ml of saturated Na₂CO₃ solution, washed with 20 ml of water, dried over MgSO₄ and evaporated to dryness. 268 mg of crude product are obtained, and are purified on a column of silica, eluting with 5/5 cyclohexane/EtOAc, and 73 mg (68%) of the expected product are thus obtained.

IR: NH: ˜3403; ˜3289 cm⁻¹ C═O: 1708; 1633 cm⁻¹ Heterocycles, aromatics and Amide II: 1611, 1573, 1539, 1505 cm⁻¹; NMR (1H, CDCl₃) 0.93 (cm) 6H: (CH₃)₂—CH 1.56 to 1.78 (cm): CH₂—CH(CH₃)₂ 3.82 (s) 3H: OCH₃ 4.46 (m) 1H: NH—CH 5.03, 5.13: (2H) AB: OCH₂Ø 5.57 (d) 1H: NH—CH 6.87, 7.47 (4H): AA′BB′ ˜7.30 (m) phenyl 7.08 (broad) 1H: H₄ 7.58 (broad) 1H: H₃ 9.14, 9.31: 2H mobile

### Example 4

### 5-(4-Methoxyphenyl)-2-thiophenecarboxylic Acid 2-[(2S)-1-oxo-3-phenyl-2-[[(phenylmethoxy)carbonyl]amino]propyl]hydrazide

The process is performed as in stage 4 of Example 3, starting with 188 mg (0.757 mM, 1 eq.) of the keto hydrazine obtained in stage 3 of Example 3, 218 mg (1.13 mM, 1.5 equivalents) of EDC, 340 mg (1.13 mM, 1.5 equivalents) of ZPheOH and 154 mg (1.13 mM, 1.5 eq.) of HOBT. 934 mg of crude product are thus obtained, and are purified on silica, eluting with 9/1 CH₂Cl₂/EtOAc. 225 mg (73%) of expected product are thus obtained in the form of two isomers C and D in respective proportions of 85% and 15%.

NMR (1H, DMSO)

The following are assigned for C: 2.82 (m), 3.08 (m): phenyl-CH₂—CH 4.38 (td): phenyl-CH₂—CH 3.81 (s): CH₃—O-phenyl 7.02, 7.67: AA′BB′ 7.46 (d), 7.83 (d): Ha and Hb

The following are assigned for D: 4.11 (broad s): phenyl-CH₂—CH 3.05 (m, 2.84 (m): phenyl-CH₂—CH

The following are assigned for C and D: 4.95 (m): phenyl-CH₂—O 7.10 to 7.40 (m): aromatic H 7.69 (masked): NH—CH—CH₂—10.28 (broad s), 10.48 (broad s): mobile H

### Example 5

### 5-[2-(Phenylmethoxy)phenyl]-2-thiophenecarboxylic Acid 2-[(2S)-4-methyl-1-oxo-2-[[(phenylmethoxy)carbonyl]amino]pentyl]hydrazide

### Stage 1: 2-(phenylmethoxy)phenylboronic Acid

1) 19.93 ml of BuLi (1.6M in hexane) (31.9 mM, 1 equivalent) is added, at −78° C. by syringe, to a solution of 8.4 g of bromo derivative (N8) (31.9 mM, 1 equivalent) in 50 ml of THF, and the mixture is stirred for 30 minutes at −78° C. A solution containing the lithiated derivative is thus obtained, and is added via a dropping funnel, at −78° C., to a solution of 30 g of B(OiPr)₃ (159.5 mM, 5 equivalents) in 50 ml of THF. The mixture is then stirred, while allowing to warm to room temperature, for 4 hours. The resulting mixture is poured into 20 ml of 3N HCl, extracted twice with 50 ml of EtOAc, washed with water, dried over MgSO₄ and evaporated to dryness. The 8.53 g of crude product are obtained, and are purified on silica, eluting with 9/1 cyclohexane/EtOAc. 3.6 g (47%) of pure product are thus obtained.

### Stage 2: Ethyl 5-[2-(phenylmethoxy)phenyl]-2-thiophenecarboxylate

The process is performed under the same experimental conditions described in stage 2 of example 3, starting with 1.0 g (4.23 mM, 2 equivalents) of the boronic acid obtained in stage 1 above, 500 mg (2.11 mM, 1 equivalent) of the bromo thiophene obtained in stage 1 of Example 3, 3.07 mg (0.266 mM, 0.125 eq.) of Pd(PØ₂)₄, 12 ml of toluene, 18 ml of EtOH and 4.6 ml of 2M Na₂CO₃. 1.79 g of crude product are thus obtained, and are purified on silica, eluting with 99/1 cyclohexane/EtOAc. 760 mg of expected product are thus obtained.

### Stage 3: 5-[2-(phenylmethoxy)phenyl]-2-thiophenecarboxylic Acid Hydrazide

4.2 g of hydrazine hydrate (84 mM, 40 eq.) are added to a solution of 713 mg of the thiophene ester obtained in stage 2 above (2.1 mM, 1 equivalent) in 20 ml of EtOH, and the mixture is stirred at reflux for 48 hours. The resulting mixture is evaporated to dryness and purified on silica, eluting with 5/5 CH₂Cl₂/EtOAc and then 9/1/0.1 CH₂Cl₂/MeOH/NH₄OH. 540 mg of expected product are thus obtained.

### Stage 4: 5-[2-(phenylmethoxy)phenyl]-2-thiophenecarboxylic Acid 2-[(2S)-4-methyl-1-oxo-2-[[(phenylmethoxy)carbonyl]amino]pentyl]hydrazide

The process is performed under the same operating conditions as those described in stage 4 of example 3, starting with 95 mg (0.293 mM, 1 equivalent) of the keto hydrazide obtained in stage 3 above, 117 mg (0.439 mM, 1.5 eq.) of ZLeuOH, 8.4 mg (0.439 mM, 1.5 eq.) of EDC and 59 mg (0.439 mM, 1.5 eq.) of HOBT. 196 mg of crude product are thus obtained, and are purified on silica, eluting with 8/2 CH₂Cl₂/EtOAc. 140 mg of expected product are thus obtained.

NMR (H, CDCl₃) 0.93 (broad s) 6H: (CH₃)₂—CH—CH₂—CH—NH 1.61 (masked), 1.73 (m) 2H: (CH₃)—CH—CH₂—CH—NH 4.43 (m) 1H: (CH₃)—CH—CH₂—CH—NH 5.45 (broad d) 1H: (CH₃)—CH—CH₂—CH—NH 5.08, 5.14, 5.22 (broad s) 4H: the O—CH₂-phenyl 6.97 (broad t) 2H: H₃ and H₄ 7.25 (m): H₂ 7.63 (dd) 1H: H₅ 7.44 (d), 7.60 (d): Ha and Hb 7.21 to 7.46 (m) 10H aromatic 8.80 (broad s), 9.16 (broad s): 2H NH

### Example 6

### 5-[2-(Phenylmethoxy)phenyl]-2-thiophenecarboxylic Acid 2-[(2S)-2-[[(1,1-dimethyl)ethoxy]carbonyl]amino]-4-methyl-1-oxopentyl]hydrazide

The process is performed under the same operating conditions as those already described in stage 4 of Example 3, starting with 336 mg (1.035 mM, 1 equivalent) of the keto hydrazide obtained in stage 3 of Example 5, 387 mg (1.55 mM, 1.5 eq.) of Boc-Leu-OH, 298 mg (1.55 mM, 1.5 eq.) of EDC and 210 mg (1.55 mM, 1.5 eq.) of HOBT. 635 mg of crude product are obtained, and are purified on a column of silica, eluting with 9/1 CH₂Cl₂/EtOAc. 439 mg of expected product are thus obtained.

NMR (1H, CDCl₃) 0.95 (d), 0.97 (d) 6H: (CH₃)₂—CH—CH₂ 1.59 (m), 1.75 (m) 3H: (CH₃)—CH—CH₂ 1.46 (s) 9H: O—C(CH₃)₃ 4.33 (broad s) 1H: NH—CH—CO 5.03 (broad d) 1H: NH—CH—CO 5.23 (AB) 2H: O—CH₂-phenyl 7.46 (d), 7.60 (d): 2H Ha and Hb 6.99 (d), 7.65 (m), 7.25 (masked) 4H: H₂, H₃, H₄ and H₅ 7.33 (t) 1H Hc′ 7.37 (broad t) 2H Hb′ 7.41 (m) 2H Ha′

### Example 7

### 5-(2-(Phenylmethoxy)phenyl)-2-thiophenecarboxylic Acid 2-((2S)-2-amino-4-methyl-1-oxopentyl)-hydrazine

The process is performed under the same operating conditions as those described in Example 10, starting with the product of Example 6 instead of the product of Example 9, and the expected product is thus obtained.

### Example 8

### 5-(1-(Naphthyl)-2-thiophenecarboxylic Acid 2-[(2S)-4-methyl-1-oxo-2-[[(phenylmethoxy)carbonyl]-amino]pentyl]hydrazide

### Stage 1: Ethyl 5-(1-naphthyl)-2-thiophenecarboxylate

The process is performed under the same operating conditions described above in stage 2 of Example 3, starting with 1.83 g of the bromo ester ethyl 5-bromo-2-thiophenecarboxylate, obtained in stage 1 of Example 3 (7.78 mM, 1 equivalent), 2.67 g (15.56 mM, 2 eq.) of naphthalene boronic acid, 1.124 g (0.97 mM, 0.125 eq.) of Pd(PØ3)₄, 44 ml of toluene, 67 ml of EtOH and 17 ml of 2M Na₂CO₃. 4.88 g of crude product are thus obtained, and are purified on a column of silica, eluting with 98/2 cyclohexane/EtOAc. 486 mg (22%) of purified product are thus obtained.

### Stage 2: 5-(1-naphthyl)-2-thiophenecarboxylic Acid Hydrazide

The process is performed under the same operating conditions as those described previously in stage 3 of Example 3, starting with 430 mg of thiophene ester 1.52 mM, 1 equivalent), obtained in stage 1 above, 2.28 g (45.6 mM, 30 eq.) of hydrazine hydrate and 15 ml of EtOH. After evaporation, the residue is purified on silica, eluting with 5/5 CH₂Cl₂/EtOAc and then 9/1/0.1 CH₂Cl₂/MeOH/NH₄OH. 306 mg (75%) of expected product are thus obtained.

### Stage 3: 5-(1-naphthyl)-2-thiophenecarboxylic Acid 2-[(2S)-4-methyl-1-oxo-2-[(phenylmethoxy)carbonyl]-amino]pentyl]hydrazide

The process is performed under the same conditions as those described previously in stage 4 of Example 3, starting with 90 mg (0.336 mM, 1 equivalent) of the thiophene keto hydrazide obtained in stage 2 above, 133 mg (0.502 mM, 1.5 equivalents) of ZLeu-OH, 96 mg (0.502 mM, 1.5 eq.) of EDC, 68 mg (0.502 mM, 1.5 eq.) of HOBT and 5 ml of DMF. 302 mg of crude product are thus obtained, and are purified on silica, eluting with 6/4 cyclohexane/EtOAc. 150 mg of expected product are thus obtained.

NMR (1H, CDCl₃) 0.94 (d), 0.96 (d): the CH₃ of iPr 1.75 (m): CH of iPr 1.75; 1.64: CH₂—CH—NH—COO— 4.47 (m): CH₂—CH—NH—COO— 5.54: CH₂—CH—NH—COO— 5.15 (d, J=12); 5.06 (d, J=12): OCH₂—C═ 7.31 (m): aromatic H 7.16 (d, J=4): H₄ 7.75 (d, J=4): H₃ 7.41 to 7.51: 4H, 7.86 (m): H₂′ and H₅′; 8.10 (d, J=8.5): H₈′; H of naphthyl; 9.20 (broad s), 9.31 (broad s): NH—NH IR in CHCl₃ NH: 3400, 3288 cm⁻¹ C═O: 1707, 1635 cm⁻¹ Heterocycle+Aromatic+Amides II=1593, 1534, 1509; 1500 cm⁻¹

### Example 9

### 5-(1-Naphthyl)-2-thiophenecarboxylic Acid 2-[(2S)-2-[[[(1,1-dimethyl)ethoxy]carbonyl]amino]-4-methyl-1-oxopentyl]hydrazide

The process is performed under the same operating conditions as those described previously in stage 4 of Example 3, starting with 247 mg of the product obtained in stage 2 of Example 8, and using Boc-Leu OH instead of ZLeuOH. 890 mg of crude product are thus obtained, and are purified on silica, eluting with 7/3 cyclohexane/EtOAc.

337 mg (76%) of expected product are thus obtained.

NMR (H, CDCl₃) 0.96 (d), 0.98 (d): 6H CH—CH₂—CH—(CH₃)₂ 1.76 (m): CH—CH₂—CH—(CH₃)₂ 1.76, 1.62: CH—CH₂—CH—(CH₃)₂ 4.39 (broad s): 1H CH—CH₂—CH—(CH₃)₂ 5.14 (broad d): 1H NH—CH—CH₂ 7.17 (d) 7.75 (d): 2H Ha and Hb 1.46 (s): 9H O—C(CH₃)₃ 7.37 to 7.53 (m): 4H H₂, H₃, H₅ and H₆ 7.85 (dd) 7.89 (dd): 2H H₁ and H₄ 8.12 (broad d): 1H H₇ 9.18 (broad s): 2H mobile H IR in CHCl₃ NH: 3420, 3288 cm⁻¹ C═O: 1703, 1690, 1635 cm⁻¹ Heterocycle+Aromatic: 1592, 1540, 1508 cm⁻¹

### Example 10

### 5-(1-Naphthyl)-2-thiophenecarboxylic Acid 2-[(2S)-2-amino-4-methyl-1-oxopentyl]hydrazide

4.5 ml of CF₃CO₂H are added to a solution of 275 mg of the product of Example 9 (—N-BOC) in 4.5 ml of CH₂Cl₂, and the mixture is stirred for 3 hours at room temperature. The resulting mixture is evaporated to dryness and the residue is taken up in 10 ml of CH₂Cl₂, washed with 10 ml of 10% NH₄OH, dried over MgSO₄ and evaporated to dryness. 177 mg (81%) of expected product are thus obtained.

NMR (H, CDCl₃) 0.97 (d) 1.00 (d): 6H the CH₃—CH—CH₂—CH 1.80 (m): CH₃—CH—CH₂—CH 1.50 (m), 1.80 (m): CH₃—CH—CH₂—CH 3.62 (dd): 1H CH₃—CH—CH₂—CH 7.17 (d), 7.74 (d): 2H Ha and Hb 7.35 to 7.53 (m): 4H H₃, H₄, H₆ and H₇ 7.86 (m): 2H H₂ and H₅ 8.13 (d): 1H H₈ IR in CHCl₃ NH/NH₂ (complex): 3365, 3221 cm⁻¹ C═O: 1687, 1635 cm⁻¹ Heterocycle+Aromatic: 1592, 1540, 1508 cm⁻¹

### Example 11

### 5-(1-Naphthyl)-2-thiophenecarboxylic Acid 2-[(2S)-2-[[[(1H-benzimidazol-2-yl)methyl]carbonyl]-amino]-4-methyl-1-oxopentyl]hydrazide

The process is performed under the same operating conditions as those described in stage 4 of Example 3, starting with 68 mg (0.178 mM, 1 equivalent) of the thiophene amine obtained in Example 10, 47 mg (0.267 mM, 1.5 eq.) of 2-((1H-benzimidazol-2-yl)methyl)carboxylic acid, 51 mg (0.267 mM, 1.5 eq.) of EDC and 36 mg (0.267 mM, 1.5 eq.) of HOBT. 118 mg of crude product are thus obtained, and are purified on silica, eluting with 97.5/2.5 CH₂Cl₂/MeOH. 19 mg (20%) of expected product are thus obtained.

NMR (1H, DMSO) 0.87 (d), 0.94 (d): 6H CH₃—CH—CH₂—CH—NH 1.73 (m): 1H CH₃—CH—CH₂—CH—NH 1.58 (t): 2H CH₃—CH—CH₂—CH—NH 4.48 (q): 1H CH₃—CH—CH₂—CH—NH 8.51 (d): 1H CH₃—CH—CH₂—CH—NH 3.86 (2×s): 2H CH₂—CO 7.07 (t): 1H H₁₁ 7.32 (t): 1H H₁₀ 7.40 (d), 7.94 (d): 2H Ha and Hb 7.46 (d): 1H H₉ 7.56 to 7.66: 4H H₃, H₄, H₆ and H₇ 8.03 (m): 2H H₂ and H₅ 8.14 (m): 1H H₈ 10.21 (broad s), 10.51 (broad s): 2H NH—NH 12.77 (broad s): 1H NH—C═N

### Example 12

### 5-(Phenylmethyl)-2-thiophenecarboxylic Acid 2-[(2S)-4-methyl-1-oxo-2-[[(phenylmethoxy)carbonyl]amino]pentyl]hydrazide

### Stage 1: Ethyl 5-(phenylmethyl)-2-thiophenecarboxylate Product G

### Ethyl 5-(1,2-diphenylethyl)-2-thiophenecarboxylate Product H

An alkylation and then an esterification are performed as follows.

1) Alkylation

12 ml of tetramethylenediamine (82 mM; 2.1 eq.) and 5 g of thiophene acid in 30 ml of THF (39 mM, 1 equivalent) are added to a solution of 41 ml of 2M LDA in THF (82 mM, 2.1 eq.) cooled to −78° C., followed, after 20 minutes, by addition of 8 g of bromophenyl (46.8 mM, 1.2 equivalents), and the mixture is stirred for 2 hours while being allowed to warm to room temperature. 300 ml of water are added (at T<20° C.) and the mixture is washed with 100 ml of Et₂O, citric acid is added to bring it to pH≈4, the mixture is extracted with 100 ml of Et₂O, washed with 100 ml of NaCl, dried over MgSO₄ and evaporated to dryness. 6.87 g of crude product are obtained.

2) Esterification.

The crude product is stirred for 8 hours in 68 ml of EtOH and 13.7 ml of H₂SO₄ at reflux at 120° C. (external temperature). The EtOH is evaporated off and the residue is extracted with 100 ml of Et₂O, washed with 50 ml of Na₂CO₃, dried over MgSO₄ and evaporated to dryness. 6.76 g of crude product are thus obtained, and are purified on silica, eluting with 99/1 n-hexane/EtOAc. 3.34 g of expected product are thus obtained in the form of a mixture of product G (monoalkyl): 2.5 g (26%) and product H (dialkyl): 0.385 g (3%).

### Stage 2: 5-(phenylmethyl)-2-thiophenecarboxylic Acid Hydrazide

The process is performed under the same conditions as those already described in stage 3 of Example 3, starting with 1.0 g (4.06 mM, 1 equivalent) of the thiophene ester obtained as product G in stage 1 above, 2.5 ml (173 mM, 43 eq.) of hydrazine hydrate and 40 ml of EtOH. The product is purified on silica, eluting with 5/5 CH₂Cl₂/EtOAc and then 9/1/0.1 CH₂Cl₂/MeOH/NH₄OH. 987 mg of expected product are thus obtained.

### Stage 3: 5-(phenylmethyl)-2-thiophenecarboxylic Acid 2-[((2S)-4-methyl-1-oxo-2-[[(phenylmethoxy)carbonyl]-amino]pentyl]hydrazide

The process is performed under the same conditions as those already described in stage 4 of Example 3, starting with 400 mg (1.72 mM, 1 equivalent) of the keto hydrazide obtained in stage 2 above, 685 mg (2.58 mM, 1.5 equivalents) of ZLeuOH, 495 mg (2.58 mM, 1.5 eq.) of EDC, 349 mg (2.58 mM, 1.5 eq.) of HOBT and 15 ml of DMF. The product is purified on silica, eluting with 6/4 cyclohexane/EtOAc. 779 mg of expected product are thus obtained (95% yield).

NMR (H, CDCl₃) 0.90 (broad s) 6H the CH₃—CH—CH₂—CH—NH 1.66 (masked) CH₃—CH—CH₂—CH—NH 1.57 (m), 1.66 (masked) CH₃—CH—CH₂—CH—NH 4.40 (m) CH₃—CH—CH₂—CH—NH 5.50 (broad d) CH₃—CH—CH₂—CH—NH 4.10 (s) 2H=C—CH₂-phenyl 5.00, 5.10 AB 2H O—CH₂-phenyl 6.71 (d), 7.46 (d) Ha and Hb 7.21 (m), 7.29 (m) 10H aromatic 8.93 (broad s), 9.21 (broad s) 2H the mobile H IR CHCl₃ NH: 3420, 3286 cm⁻¹ C═O: 1706, 1630 cm⁻¹ Heterocycle+Aromatic+Amide II: 1587, 1540, 1509, 1497 cm⁻¹.

### Example 13

### 5-[(Dimethyl)amino]-2-thiophenecarboxylic Acid 2-[(2S)-4-methyl-1-oxo-2-[[(phenylmethoxy)carbonyl]amino]pentyl]hydrazide

### Stage 1: Ethyl 5-(dimethylamino)-2-thiophenecarboxylate

2.0 g of the bromo ester obtained in stage 1 of Example 3 (8.5 mM, 1 equivalent) in 60 ml of DMF and 60 ml of 33% dimethylamine in ethanol are stirred at 165° C. (external temperature) overnight. The solvents are evaporated off and the residue is taken up in CH₂Cl₂, washed with ? ml of 10% NaHCO₃, dried over MgSO₄ and evaporated to dryness. 1.668 g of crude product are thus obtained, and are purified on silica, eluting with 8/2 cyclohexane/EtOAc. 632 mg (37%) of expected product are thus obtained.

### Stage 2: 5-(dimethylamino)-2-thiophenecarboxylic Acid Hydrazide

The process is performed under the same operating conditions as those described previously in stage 3 of Example 3. 200 mg of the thiophene ester (1 mM, 1 eq.) obtained in stage 1 above are dissolved in 10 ml of EtOH. 1.5 ml of hydrazine hydrate (30 mM, 30 equivalents) are added and the mixture is stirred at reflux for 2 hours. The resulting mixture is evaporated to dryness and 195 g of the expected product are thus obtained.

### Stage 3: 5-[(dimethyl)amino]-2-thiophenecarboxylic Acid 2-[(2S)-4-methyl-1-oxo-2-[[(phenylmethoxy)carbonyl]amino]pentyl]hydrazide

The process is performed under the same operating conditions as those described previously in stage 4 of Example 3, starting with 100 mg (0.540 mM, 1 equivalent) of the keto hydrazide obtained in stage 2 above, 186 mg (0.701 mM, 1.3 eq.) of ZLeuOH, 135 mg (0.701 mM, 1.3 eq.) of EDC, 95 mg (0.701 mM, 1.3 eq.) of HOBT and 5 ml of DMF. 671 mg of crude product are thus obtained, and are purified on silica, eluting with 5/5 cyclohexane/EtOAc. 141 mg (61%) of expected product are thus obtained.

NMR (H, CDCl₃) 0.92 (m): 6H the (CH₃)₂—CH 1.53 to 1.78: (CH₃)₂CH—CH₂ 2.99 (s): 6H the NCH₃ 4.40 (m): 1H CH—NH 5.50 (d): CH—NH 5.03, 5.13: 2H AB OCH₂Ø 5.77 (d): 1H H₃ 7.32 (broad s): 5H of phenyl 7.40 (d): 1H H₄ 8.49 (broad s), 9.14 (broad s): 2H mobile IR (H, CHCl₃) NH: 3408, 3290 cm¹ C═O: 1710, 1623 cm¹ Heterocycle+Aromatic+Amide II: 1546, 1501 cm¹

### Example 14

### 5-[(Dimethyl)amino]-2-thiophenecarboxylic acid 2-[(2S)-1-oxo-3-phenyl-2-[[(phenylmethoxy)carbonyl]amino]propyl]hydrazide

The process is performed under the same operating conditions as those used previously in stage 4 of Example 3, starting with 81 mg (0.437 mM, 1 equivalent) of the keto thiophene hydrazide obtained in stage 2 of Example 13, 197 mg (0.656 mM, 1.5 eq.) of ZPheOH, 126 mg (0.656 mM, 1.5 eq.) of EDC, 89 mg (0.656 mM, 1.5 eq.) of HOBT and 5 ml of DMF. 452 mg of crude product are thus obtained, and are purified on silica, eluting with 5/5 cyclohexane/EtOAc. 130 mg (64%) of expected product are thus obtained.

NMR (H, CDCl₃) 2.88 (dd), 3.14 (masked): phenyl-CH₂—CH 3.00 (s): 6H the CH₃—N-4.44 (m): 1H phenyl-CH₂—CH—NH 4.99 (s): 2H phenyl-CH₂—O 5.94 (d), 7.61 (d): 2H Hₐ and H_{b} 7.13 to 7.46 (m): 10H aromatics 9.79 (broad s), 9.91 (broad s): mobile H

### Example 15

### 5-[(Phenylmethyl)amino]-2-thiophenecarboxylic Acid 2-[(2S)-4-methyl-1-oxo-2-[[(phenylmethoxy)-carbonyl]amino]pentyl]hydrazide

### Stage 1: Ethyl 5-nitro-2-thiophenecarboxylate

A solution of 8.72 g of 5-nitro-2-thiophenecarboxylic acid (50.4 mM, 1 equivalent) in 18 ml of SOCl₂ (252 mM, 5 eq.) is refluxed for 3 hours. The SOCl₂ is evaporated off, being taken up in twice 50 ml of toluene. The resulting material is taken up in 35 ml of EtOH and the solution is refluxed for 3 hours. The resulting mixture is evaporated to dryness and washed with 25 ml of 10% NaHCO₃, extracted with 50 ml of EtOAc, dried over MgSO₄ and evaporated to dryness. 9.69 g (96%) of expected product are thus obtained.

### Stage 2: Ethyl 5-amino-2-thiophenecarboxylate

A suspension of 1.0 g (4.97 mM, 1 equivalent) of the product obtained in stage 1 above and 1.64 g of iron powder (29.82 mM, 6 equivalents) in 8 ml of glacial acetic acid is stirred at 75° C. for 3 hours. 10 ml of water are added, the mixture is filtered, the filtrate is neutralized to pH≈8 with Na₂CO₃, extracted with 25 ml of EtOAc, dried over MgSO₄ and evaporated to dryness. 917 mg of crude product are thus obtained, and are purified on silica, eluting with 9/1 CH₂Cl₂/EtOAc. 619 mg (73%) of expected product are thus obtained.

### Stage 3: Ethyl 5-[bis(phenylmethyl)amino]-2-thiophenecarboxylate Product E

### Ethyl 5-[(phenylmethyl)amino]-2-thiophenecarboxylate Product F

A solution of 470 mg of the thiophene amine (2.75 mM, 1 equivalent) obtained in stage 2 above, 2,6-lutidine (3.84 mM, 1.4 equivalents) and BrCH₂Ø bromomethylphenyl (3.57 mM, 1.3 equivalents) in 10 ml of DMF is stirred at 60° C. for 36 hours. 10 ml of water are added and the mixture is extracted with 25 ml of Et₂O, dried over MgSO₄ and evaporated to dryness. 940 mg of crude product are thus obtained, and are purified on silica, eluting with 8/2 cyclohexane/EtOAc. 182 mg (19%) of product E and 269 mg (37%) of product F are thus obtained.

NMR (H, CDCl₃) Product E 1.31 (t): 3H CH₃—CH₂—O—CO 4.26 (q): 2H CH₃—CH₂—O—CO 4.56 (broad s): 4H the phenyl-CH₂—N 5.91 (d), 7.50 (d): 2H Hₐ and H_{b} 7.20 to 7.26 (m): 4H, 7.27 to 7.38 (m) 6H: aromatic H NMR (H, CDCl₃) Product F 1.33 (t): 3H CH₃—CH₂—O—CO 4.27 (q): 2H CH₃—CH₂—O—CO 4.36 (d): 2H phenyl-CH₂—NH 4.75 (broad s): 1H phenyl-CH₂—NH 5.97 (d), 7.49 (d): 2H Hₐ and H_{b} 7.36 (m): 5H aromatic

### Stage 4: 5-[(phenylmethyl)amino]-2-thiophenecarboxylic Acid Hydrazide

The process is performed under the same conditions as those already described in stage 3 of Example 3, starting with 150 mg (0.57 mM, 1 equivalent) of the thiophene ester product E obtained in stage 3 above, 1.1 g (23 mM, 40 eq.) of hydrazine hydrate and 5 ml of EtOH. The product is purified on silica, eluting with 5/5 CH₂Cl₂/EtOAc and then 9/1/0.1 CH₂Cl₂/MeOH/NH₄OH. 119 mg (84%) of expected product are thus obtained.

### Stage 5: 5-[(phenylmethyl)amino]-2-thiophenecarboxylic Acid 2-[(2S)-4-methyl-1-oxo-2-[[(phenylmethoxy)carbonyl]amino]pentyl]hydrazide

The process is performed under the same conditions as those already described in stage 4 of Example 3, starting with 119 mg (0.481 mM, 1 equivalent) of the keto hydrazide obtained in stage 4 above, 191 mg (0.721 mM, 1.5 eq.) of ZLeuOH, 138 mg (0.721 mM, 1.5 eq.) of EDC, 98 mg (0.721 mM, 1.5 eq.) of HOBT and 5 ml of DMF. 254 mg of crude product are thus obtained, and are purified on silica, eluting with 7/3 CH₂Cl₂/EtOAc. 162 mg (68%) of expected product are thus obtained.

NMR (H, CDCl₃) 0.91 (m): 6H the (CH₃)₂—CH—CH₂—CH—NH 1.69 (masked): (CH₃)₂—CH—CH₂—CH—NH 1.58 (m): (CH₃)₂—CH—CH₂—CH—NH 4.38 (m): 1H (CH₃)₂—CH—CH₂—CH—NH 5.48 (broad d): 1H (CH₃)₂—CH—CH₂—CH—NH 4.30 (d): 2H phenyl-CH₂—NH 4.80 (t): 1H phenyl-CH₂—NH 5.01, 5.11: AB 2H phenyl-CH₂—O 7.30 to 7.40 (m): 10H aromatic 5.90 (d), 7.33 (masked): Ha and Hb 8.53 (broad d), 9.10 (broad d): 2H the mobile H IR CHCl₃—NH: 3409, 3288 cm⁻¹ >=O: 1708, 1626 cm⁻¹ Conjugated system+Aromatic+Amide II: 1585, 1485 cm⁻¹

### Example 16

### 5-[Bis(phenylmethyl)amino]-2-thiophenecarboxylic Acid 2-[(2S)-4-methyl-1-oxo-2-[[(phenylmethoxy)carbonyl]amino]pentyl]hydrazide

### Stage 1: 5-[bis(phenylmethyl)amino]-2-thiophenecarboxylic Acid Hydrazide

The process is performed as in stage 4 of Example 15, starting with 180 mg (0.512 mM, 1 equivalent) of the thiophene ester product F obtained in stage 3 of Example 15, 3 ml (60 mM, 120 equivalents) of NH₂NH₂.H₂O and 16 ml of EtOH. The crude product is purified on silica, eluting with 5/5 CH₂Cl₂/EtOAc and then 9/1/0.1 CH₂Cl₂/MeOH/NH₄OH. 96 mg (56%) of expected product are thus obtained.

### Stage 2: 5-[bis(phenylmethyl)amino]-2-thiophenecarboxylic Acid 2-[(2-S)-4-methyl-1-oxo-2-[[(phenylmethoxy)carbonyl]amino]pentyl]hydrazide

The process is performed under the same operating conditions as those already described in stage 4 of Example 3, starting with 96 mg (0.284 mM, 1 equivalent) of the keto hydrazide obtained in stage 1 above, 113 mg (0.426 mM, 1.5 equivalents) of ZLeuOH, 82 mg (0.426 mM, 1.5 eq.) of EDC, 58 mg (0.426 mM, 1.5 eq.) of HOBT and 5 ml of DMF. 205 mg of crude product are thus obtained, and are purified on silica, eluting with 9/1 CH₂Cl₂/EtOAc. 129 mg (78%) of expected product are thus obtained.

NMR (H, CDCl₃) 0.90 (broad s): 6H (CH₃)₂—CH—CH₂—CH—NH 1.69 (masked): 1H (CH₃)₂—CH—CH₂—CH—NH 1.56 (m): 2H (CH₃)₂—CH—CH₂—CH—NH 4.37 (m): 1H (CH₃)₂—CH—CH₂—CH—NH 5.44 (broad d): 1H (CH₃)₂—CH—CH₂—CH—NH 4.52 (broad s): 4H N—CH₂-Ø 5.02 (d), 5.11 (d): 2H O—CH₂-Ø 5.85 (d): 1H H4 7.20 (dd): 4H Hₐ 7.26 to 7.35: 11H H_{b}, H_{c}, H_{d}, Hₑ, H_{f} 7.32 (masked): 1H H_{g} 8.36 (broad s), 9.05 (broad s): 2H mobile H

### Example 17

### 5-[[[3-(4-Methoxyphenoxy)phenyl]methyl]-(phenylmethyl)amino]-2-thiophenecarboxylic Acid 2-[(2S)-4-methyl-1-oxo-2-[[(phenylmethoxy)carbonyl]-amino]pentyl]hydrazide

### Stage 1: Ethyl 5-[[3-[(4-methoxy)phenoxy]phenyl]-(phenylmethyl)amino]-2-thiophenecarboxylate

The alkylation of the benzyl amine is performed as follows.

A solution of 110 mg (0.420 mM, 1 equivalent) of the thiophene ester product E obtained in stage 3 of Example 15, 160 mg (0.547 mM, 1.3 eq.) of 3-((4-methoxyphenoxy)phenyl)methyl bromide and 63 mg (0.586 mM, 1.4 eq.) of 2,6-lutidine in 5 ml of DMF is stirred at 80° C. for 24 hours. 25 ml of Et₂O are added, and the mixture is washed with 25 ml of water, dried over MgSO₄ and evaporated to dryness. 333 mg of crude product are obtained, and are purified on silica, eluting with 9/1 cyclohexane/EtOAc. 108 mg (54%) of expected product are thus obtained.

### Stage 2: 5-[[3-[(4-methoxy)phenoxy]phenyl](phenylmethyl)amino]-2-thiophenecarboxylic Acid Hydrazide

The process is performed under the same operating conditions as those already described in stage 3 of Example 3, starting with 93 mg (0.196 mM, 1 equivalent) of the thiophene ester obtained in stage 1 above, 1.4 ml (28.8 mM, 147 eq.) of hydrazine hydrate and 5 ml of EtOH. The product is purified on a column of silica, eluting with 5/5 CH₂Cl₂/EtOAc and then 9/1/0.1 CH₂Cl₂/MeOH/NH₄OH. 49 mg (54%) of expected product are thus obtained.

### Stage 3: 5-[[[3-(4-methoxyphenoxy)phenyl]methyl]-(phenylmethyl)amino]-2-thiophenecarboxylic Acid 2-[(2S)-4-methyl-1-oxo-2-[[(phenylmethoxy)carbonyl]-amino]pentyl]hydrazide

The process is performed under the same conditions as those already described in stage 4 of Example 3, starting with 48 mg (0.104 mM, 1 equivalent) of the keto hydrazide obtained in stage 2 above, 42 mg (0.157 mM, 1.5 equivalents) of ZLeuOH, 30 mg (0.157 mM, 1.5 eq.) of EDC, 21 mg (0.157 mM, 1.5 eq.) of HOBT and 5 ml of DMF. 81 mg of crude product are thus obtained, and are purified on silica, eluting with 5/5 CH₂Cl₂/EtOAc. 60 mg (81%) of expected product are thus obtained.

NMR (H, CDCl₃) 0.91 (broad s): 6H (CH₃)₂—CH—CH₂—CH—NH 1.56 (m): 1H (CH₃)₂—CH—CH₂—CH—NH 1.69 (m): 2H (CH₃)₂—CH—CH₂—CH—NH 4.36 (m): 1H (CH₃)₂—CH—CH₂—CH—NH 5.36 (broad d): 1H (CH₃)₂—CH—CH₂—CH—NH 3.80 (s): 3H CH₃—O-phenyl 4.47 (s), 4.51 (s): 4H the N—CH₂-phenyl 5.04, 5.16: AB 2H phenyl-CH₂—O 5.85 (d), 7.31 (masked): 2H Hₐ and H_{b} 6.82, 6.94: AA.BB′ O-phenyl-O— 6.76 to 6.96 (m): 3H, 7.15 to 7.34 (m) 11H: aromatic H 8.24 (broad s), 8.91 (broad s): 2H the NH

### Example 18

### 5-[(1,2-Diphenyl)ethyl]-2-thiophenecarboxylic Acid 2-[(2S)-4-methyl-1-oxo-2-[[(phenylmethoxy)carbonyl]amino]pentyl]hydrazide

### Stage 1: 5-(1,2-diphenylethyl)-2-thiophenecarboxylic Acid Hydrazide

The process is performed as in stage 2 of Example 12, starting with 315 mg (0.936 mM, 1 equivalent) of thiophene ester, 2.4 g (48 mM, 52 equivalents) of hydrazine hydrate and 15 ml of EtOH. The product is purified on silica, eluting with 5/5 CH₂Cl₂/EtOAc and then 9/1/0.1 CH₂Cl₂/MeOH/NH₄OH. 135 mg (50%) of expected product are thus obtained.

### Stage 2: 5-[(1,2-diphenyl)ethyl]-2-thiophenecarboxylic Acid 2-[(2S-4-methyl-1-oxo-2-[[(phenylmethoxy)carbonyl]amino]pentyl]hydrazide

The process is performed under the same conditions as those already described in stage 4 of Example 3, starting with 120 mg (0.372 mM, 1 equivalent) of the keto hydrazide obtained in stage 1 above, 148 mg (0.558 mM, 1.5 equivalents) of ZLeuOH, 1.07 mg (0.558 mM, 1.5 eq.) of EDC, 75 mg (0.558 mM, 1.5 eq.) of HOBT and 5 ml of DMF. The product is purified on a column of silica, eluting with 9/1 CH₂Cl₂/EtOAc. 161 mg (76%) of expected product are thus obtained.

NMR (H, CDCl₃) 0.90 (CH₃)—CH—CH₂—CH—NH 1.39 (m) (CH₃)—CH—CH₂—CH—NH 1.57 (m) (CH₃)—CH—CH₂—CH—NH 4.39 (masked) (CH₃)—CH—CH₂—CH—NH 5.44 (CH₃)—CH—CH₂—CH—NH 3.34 (m) Ø-CH₂—CH 4.40 (t) Ø-CH₂—CH 5.06 (AB) OCH₂ Ø 6.88 (d), 7.40 the H of the thiophene 7.10 to 7.35 aromatic H 8.73 (s), 9.22 (s) CO—NH NH—CO

### Example 19

### 5-[1-[(Phenylmethoxy)imino]ethyl]-2-thiophenecarboxylic Acid 2-[(2S)-4-methyl-1-oxo-2-[[(phenylmethoxy)carbonyl]amino]pentyl]hydrazide

### Stage 1: 5-[1-(phenylmethoxyimino)ethyl]-2-thiophenecarboxylic Acid

0.94 g of benzylhydroxylamine is added to a solution of 7 ml of pyridine containing 0.5 g of an appropriate acid (N31). The medium is maintained at 100° C. for 20 hours. The solution is then evaporated under vacuum and the residue is taken up in 25 ml of 2N HCl solution and 15 ml of ethyl acetate. This mixture is separated by settling of the phases, and the organic phase is dried over MgSO₄, filtered and concentrated. 0.430 g of the expected product is recovered, and is used without further purification for the following synthesis.

### Stage 2: 5-[1-[(phenylmethoxyimino)ethyl]-2-thiophenecarboxylic Acid 2-[(2S)-4-methyl-1-oxo-2-[[(phenylmethoxy)carbonyl]amino]pentyl]hydrazide

The process is performed under the same operating conditions as those described in stage 4 of Example 3 for the coupling of acid and ZLeu. 0.5 g of the expected product is thus recovered, and is purified by chromatography, eluting with a dichloromethane/methanol solution (95/5).

NMR (1H, DMSO): 1.49 (s, 9H); 2.19 (s, 3H); 2.27 (s, 3H); 5.18 (s, 2H); 5.30 (s, 2H); 6.65 (broad s, 1H); 7.09 (d, 1H); 7.29 to 7.45 (m, 5H); 7.32 (broad s, 1H); 7.50 (d, 1H).

### Example 20

### 5-[1-[[(Phenylmethoxy)carbonyl]amino]-ethyl]-2-thiophenecarboxylic Acid 2-[[(9H-fluoren-9-yl)methoxy]carbonyl]hydrazide

### Stage 1: 5-(1-aminoethyl)-2-thiophenecarboxylic Acid 2-[[(9H-fluoren-9-yl)methoxy]carbonyl]hydrazide

The process is performed as in stage 3 of Example 1, starting with the product of Example 2. After working up the reaction medium, 0.64 g of salt of the amine is obtained. This salt is used immediately in the following step.

### Stage 2: 5-[1-[[(phenylmethoxy)carbonyl]amino]-ethyl]-2-thiophenecarboxylic acid 2-[[(9H-fluoren-9-yl)methoxy]carbonyl]hydrazide

Under nitrogen, to a solution of 5 ml of THF containing 0.64 g of trifluoroacetate salt of the amine obtained in stage 1 above. The reaction medium is cooled to about 0-5° C. (ice-salt bath). 0.22 g of benzyl chloroformate is then added dropwise thereto. After 30 minutes at this temperature, the medium is maintained at room temperature for 4 hours. The resulting mixture is evaporated under vacuum and the residue is taken up in 25 ml of CH₂Cl₂ and washed with 15 ml of 1N HCl solution. The CH₂Cl₂ solution is separated out by settling of the phases, and concentrated. The residue obtained is purified by chromatography, eluting with a dichloromethane/methanol solution (96/4). 0.40 g of the expected product is thus obtained.

NMR (1H, CDCl₃): 1.56 (d, 3H); 4.26 (t, 1H); 4.47 (d, 2H); 5.13 (m, 2H); 6.91 (m, 2H); 7.34 (m, 9H); 7.59 (d, 2H); 7.75 (d, 2H); 8.17 (broad s, 1H).

### Example 21

### 5-[1-[[(Phenylmethoxy)carbonyl]amino]ethyl]-2-thiophenecarboxylic Acid 2-[(2S)-4-methyl-1-oxo-2-[[(phenylmethoxy)carbonyl]amino]pentyl]hydrazide

### Stage 1: 5-[1-[[(phenylmethoxy)carbonyl]amino]-ethyl]-2-thiophenecarboxylic Acid Hydrazide

Deprotection of the Fmoc carbonate is performed as follows.

2 ml of piperidine are added to a solution of 10 ml of acetonitrile at room temperature containing 0.35 g of the thiophene derivative obtained in Example 20. The medium is stirred for 2 hours at room temperature and then concentrated under vacuum. The residue is purified by chromatography, eluting with a CH₂Cl₂/MeOH solution (95/5). 0.150 g of the expected product are thus obtained.

### Stage 2: 5-[1-[[(phenylmethoxy)carbonyl]amino]-ethyl]-2-thiophenecarboxylic Acid 2-[(2S)-4-methyl-1-oxo-2-[[(phenylmethoxy)carbonyl]amino]pentyl]hydrazide

An acid-amine coupling is performed, under the operating conditions described in stage 4 of Example 3. 0.050 g of the expected product is thus obtained, and is purified by chromatography, eluting with a dichloromethane/MeOH solution (96/4).

### Example 22

### 5-[3-methyl-1-[(phenylmethoxy)carbonyl]-amino]butyl]-2-thiophenecarboxylic Acid 2-[(2S)-4-methyl-1-oxo-2-[[(phenylmethoxy)carbonyl]amino]pentyl]-hydrazide

### Stage 1: Methyl 5-(1-hydroxy-3-methylbutyl)-2-thiophenecarboxylate

2.5 g (19.5 mM, 1 eq.) of 2-thiophenecarboxylic acid dissolved in 20 ml of THF are added to a solution of 22.5 ml of LDA (2M in THF) (44.87 nM, 2.3 eq.) cooled to −78° C., 1.84 g of isovaleraldehyde (21.46 mM, 1 eq.) are added and the mixture is stirred at room temperature for 2 hours. H₂O (150 ml) is added, the mixture is washed with 50 ml of Et₂O, citric acid is added to pH≈4, the resulting mixture is extracted with 50 ml of CH₂Cl₂ and the organic extracts are dried over MgSO₄. 3.37 g of crude product are thus obtained, and are taken up in 50 ml of CH₂Cl₂. 60 ml of CH₂N₂/CH₂Cl₂ (12 g/l) (1.65 equivalents) are added and the mixture is stirred at room temperature for 3 hours and evaporated to dryness. 1.67 g of crude product are thus obtained, and are purified on silica, eluting with 9/1 n-hexane/EtOAc. 855 mg (37%) of expected product are thus obtained.

### Stage 2: Methyl 5-(3-methyl-1-oxobutyl)-2-thiophenecarboxylate

A suspension of 787 mg of the thiophene ester obtained in stage 1 above, 2.36 g of MnO₂ (3 parts by weight) and 15 ml of CH₂Cl₂ is stirred at reflux for 5 hours. The resulting mixture is filtered and the filtrate is evaporated to dryness. 722 mg of crude product are thus obtained.

### Stage 3: Methyl 5-(3-methyl-1-hydroxyiminobutyl)-2-thiophenecarboxylate

319 mg of hydroxylamine hydrochloride (4.59 mM, 2 eq.) are added to a solution of 520 mg of the thiophene (2.29 mM, 1 equivalent) obtained in stage 2 above, in 5 ml of pyridine, and the mixture is stirred at reflux for 2 hours. The pyridine is evaporated off, 50 ml of 2N HCl are added, the resulting mixture is extracted with 50 ml of EtOAc and the organic extracts are dried over MgSO₄ and evaporated to dryness. 554 mg of expected product are thus obtained.

### Stage 4: Methyl 5-(1-amino-3-methylbutyl)-2-thiophenecarboxylate

A suspension of 550 mg of the thiophene hydroxy amine (2.28 mM, 1 equivalent) obtained in stage 3 above, 1.49 g of zinc powder (22.8 mM, 10 equivalents) in 10 ml of CF₃CO₂H. The CF₃CO₂H is evaporated off and the residue is taken up in 25 ml of MeOH, filtered and evaporated to dryness. This residue is taken up in 50 ml of CH₂Cl₂, extracted with 25 ml of 1N HCl, brought to pH≈8 with 10% NaHCO₃, extracted with 50 ml of EtOAc; dried over MgSO₄ and evaporated to dryness. 316 mg (61%) of expected product are thus obtained.

### Stage 5: Methyl 5-[1-[[(phenylmethoxy)carbonyl]-amino]-3-methylbutyl]-2-thiophene Carboxylate

238 mg of TEA (2.365 mM, 1.7 equivalents) and 403 mg of ClCO₂CH₂Ø (2.365 mM, 1.7 eq.) are added to a solution of 315 mg of the thiophene amine (1.39 mM, 1 eq.), obtained in stage 4 above, in 10 ml of THF cooled to 0° C. The THF is evaporated off and the residue is taken up in 50 ml of CH₂Cl₂, washed with 25 ml of 2N HCl, dried over MgSO₄ and evaporated to dryness. 548 mg of crude product are thus obtained, and are purified on silica, eluting with 9/1 n-hexane/EtOAc. 312 mg (62%) of expected product are thus obtained.

### Stage 6: 5-[1-[[(phenylmethoxy)carbonyl]amino]-3-methylbutyl]-2-thiophenecarboxylic Acid

A solution of 277 mg of the thiophene ester (0.766 mM, 1 equivalent), obtained in stage 5 above, and 48 mg of LiOH (1.148 mM, 1.5 eq.) in 6 ml of THF and 3 ml of H₂O is stirred overnight at 40° C.

The THF is evaporated off, the residue is washed with 25 ml of CH₂Cl₂ and extracted with 10 ml of NaHCO₃, brought to pH≈1 with 2N HCl, extracted three times with 25 ml of EtOAc, washed with 25 ml of water, dried over MgSO₄ and evaporated to dryness. 280 mg of expected product are thus obtained.

### Stage 7: 5-[3-methyl-1-[(phenylmethoxy)carbonyl]-amino]butyl]-2-thiophenecarboxylic Acid 2-[(2S)-4-methyl-1-oxo-2-[[(phenylmethoxy)carbonyl]amino]pentyl]-hydrazide

The process is performed under the same conditions as those already described in stage 4 of Example 3, starting with 270 mg of the thiophene acid obtained in stage 6 above (0.778 mM, 1 equivalent), 310 mg (1.1 mM, 1.42 eq.) of the keto hydrazine, 164 mg (0.855 mM, 1.1 eq.) of EDC, 115 mg (0.855 mM, 1.1 eq.) of HOBT and 10 ml of DMF. 360 mg of crude product are thus obtained, and are purified on silica, eluting with 95/5 CH₂Cl₂/MeOH. 310 mg (67%) of expected product are thus obtained in the form of a mixture of diastereoisomers.

NMR (1H, DMSO) 0.89 (m) 12H (CH₃)₂—CH—CH₂ 1.71 (m) 2H (CH₂)₂—CH—CH₂ 1.54 (m): 4H (CH₂)₂—CH—CH₂ 4.16 (ddd): 1H CO—CH—NH 7.53 (broad d): 1H CO—CH—NH 4.83 (ddd): 1H C═C—CH—NH—7.48 (broad d): 1H C═C—CH—CH 5.03 (broad s): 4H O—CH₂-Ø 7.00 (d) and 7.66 (d): 2H Ha and Hb 7.27 to 7.39 (m): 10H aromatic 10.05 (broad s) and 10.32 (broad s): 2H CO—NH—NH—CO

### Example 23

### 5-[3-methyl-1-[[(phenylmethoxy)carbonyl]-amino]butyl]-2-thiophenecarboxylic Acid 2-[(2S)-4-methyl-1-oxo-2-[[(phenylmethoxy)carbonyl]amino]pentyl]-hydrazide Isomer A

### Example 24

### 5-[3-Methyl-1-[[(phenylmethoxy)carbonyl]-amino]butyl]-2-thiophenecarboxylic Acid [(2S)-4-methyl-1-oxo-2-[[(phenylmethoxy)carbonyl]amino]pentyl]hydrazide Isomer B

After purification and separation of 50 mg of the product of Example 22 on a chiral column, isomer A, which constitutes example 23, and isomer B, which constitutes Example 24, are obtained.

NMR Ex. 23 isomer A 0.89 (m) 12H: the (CH₃)₂—CH—CH₂ 1.43 to 1.78 (m) 6H: the (CH₃)₂—CH—CH₂ 4.15 (ddd) 1H: CO—CH—NH 7.52 (broad d) 1H: CO—CH—NH 4.83 (ddd) 1H: ═C—CH—NH 7.96 (broad d) 1H: ═C—CH—NH 5.03 (broad s) 4H: the O—CH₂-phenyl 7.34 (m) 10H: the O—CH₂-phenyl 6.98 (d), 7.63 (d) 2H: Ha and Hb 10.07 (broad s) 2H: NH—NH NMR Ex. 24 isomer B 0.89 (m) 12H: the (CH₃)₂—CH—CH₂ 1.46 to 1.78 (m) 6H: the (CH₃)₂—CH—CH₂ 4.13 (ddd) 1H: CO—CH—NH 7.53 (broad d) 1H: CO—CH—NH 4.82 (ddd) 1H: ═C—CH—NH 7.93 (broad d) 1H: ═C—CH—NH 5.04 (broad s) 4H: the O—CH₂-phenyl 7.35 (m) 10H: the O—CH₂-phenyl 6.96 (d), 7.56 (broad s) 2H: Ha and Hb 10.02 (broad s) 10.37 (broad s) 2H: NH—NH

### Example 25

### Pharmaceutical Composition

Tablets corresponding to the following formula were prepared:

| | |
|---|---|
| Product of Example 1 | 0.2 g |
| Excipient for a finished tablet weighing | 1 g |
| (details of the excipient: lactose, talc, | |
| starch, magnesium stearate). | |

1. A compound of formula (I): 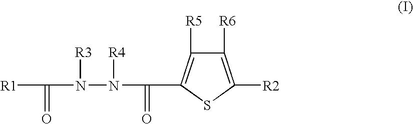
in which:
R1 is a linear or branched alkyl or alkoxy radical containing up to 8 carbon atoms substituted either with a monocyclic radical or consisting of carbocyclic or heterocyclic, saturated or unsaturated fused rings that are up to 14-membered, containing one or more identical or different hetero atoms, O, N, NH or S, such a monocyclic radical or a radical consisting of fused rings itself being optionally substituted as indicated below,
or with an amino radical that is itself optionally substituted with a radical —C(O)—O—R7 or —C(O)—R7,
R2 is:
a) a linear or branched alkyl radical containing up to 8 carbon atoms optionally substituted with one or more radicals chosen from the radicals —NH—C(O)—O—R7, radicals —NH—C(O)-alk-NH—C(O)—O—R7 and monocyclic radicals or radicals consisting of carbocyclic or heterocyclic, saturated or unsaturated fused rings that are up to 14-membered, containing one or more identical or different hetero atoms, O, N, NH or S, such monocyclic radicals or radicals consisting of fused rings, themselves being optionally substituted as indicated below,
b) a phenyl radical optionally substituted with a linear or branched alkoxy radical containing up to 6 carbon atoms or an arylalkoxy radical, these alkoxy and arylalkoxy radicals themselves being optionally substituted as indicated below,
c) a naphthyl radical optionally substituted as indicated below,
d) an amino radical optionally substituted with one or two radicals, which may be identical or different, chosen from linear or branched alkyl radicals containing up to 6 carbon atoms, radicals —C(O)-alk-NH—C(O)—O—R7 and arylalkyl radicals, themselves optionally substituted with an alkoxyphenoxy radical in which the linear or branched alkoxy radical contains up to 6 carbon atoms, these alkyl, arylalkyl, alkoxy and alkoxyphenoxy radicals themselves being optionally substituted as indicated below,
e) >=N—O—R8,
R3 and R4, which may be identical or different, are hydrogen or an alkyl, arylalkyl or aryl radical, in which the alkyl radicals are linear or branched, containing up to 6 carbon atoms, and the aryl radicals contain up to 10 carbon atoms, these alkyl, arylalkyl and aryl radicals being optionally substituted with a halogen, a hydroxyl radical or a linear or branched alkyl or alkoxy radical containing up to 4 carbon atoms,
R5 and R6, which may be identical or different, are chosen from hydrogen, a hydroxyl radical and linear or branched alkyl and alkoxy radicals containing up to 6 carbon atoms, these alkyl and alkoxy radicals themselves being optionally substituted as indicated below,
R7 is a linear or branched alkyl radical containing up to 6 carbon atoms, optionally substituted with an optionally substituted aryl or heteroaryl (indazolyl) radical,
R8 is hydrogen or a linear or branched alkyl radical containing up to 6 carbon atoms optionally substituted with an aryl (phenyl) radical, it being understood that all alkyl and alkoxy radicals, monocyclic radicals or radicals consisting of fused rings, aryl, heteroaryl, arylalkyl, arylalkoxy and alkoxyphenoxy radicals indicated above as being optionally substituted, are optionally substituted with one or more radicals chosen from halogen; hydroxyl radicals; alkyl, alkenyl, alkylthio or alkoxy containing up to 6 carbon atoms; up to 6-membered cycloalkyl; acyl containing up to 7 carbon atoms; cyano; nitro; free, salified or esterified carboxyl; tetrazolyl; indazolyl; —NH2, —NH(alk) or —N(alk)(alk); —SO2-NH—CO—NHR5 in which R5 is alk or phenyl; —C(O)—NH2, —C(O)—NH(alk), —C(O)—N(alk)(alk), —NH—C(O)-(alk), —N(alk)-C(O)-(alk); and phenyl itself optionally substituted with an alkyl or alkoxy radical containing up to 4 carbon atoms,
it being understood that alk is a linear or branched alkyl radical containing up to 4 carbon atoms,
said compounds of formula (I) being in all the possible racemic, enantiomeric and diastereoisomeric isomer forms, or the addition salts with mineral and organic acids or with mineral and organic bases of said compounds of formula (I).

2. The compound of formula (I) as defined in claim 1, corresponding to formula (Ia): 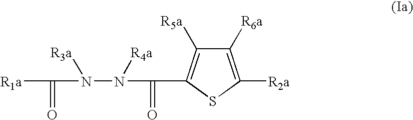
in which:
R1a is a linear or branched alkyl or alkoxy radical containing up to 8 carbon atoms substituted with a phenyl radical, a radical consisting of saturated or unsaturated carbocyclic fused rings that are up to 14-membered and an amino radical that is itself optionally substituted with a radical —C(O)—O—R7a or —C(O)—R7a, this phenyl radical and these radicals consisting of saturated or unsaturated carbocyclic fused rings being optionally substituted as indicated below,
R2a is:
a) a linear or branched alkyl radical containing up to 6 carbon atoms optionally substituted with one or more radicals chosen from the radicals NH—C(O)—O—R7a and —NH—C(O)-alk-NH—C(O)—O—R7, a phenyl radical and saturated or unsaturated heterocyclic radicals that are up to 10-membered,
b) a phenyl radical optionally substituted with a linear or branched alkoxy radical containing up to 6 carbon atoms or a benzyloxy radical,
c) a naphthyl radical,
d) an amino radical optionally substituted with one or two radicals, which may be identical or different, chosen from linear or branched alkyl radicals containing up to 4 carbon atoms, radicals —C(O)-alk-NH—C(O)—O—R7a and a benzyl radical, itself optionally substituted with an alkoxyphenoxy radical in which the alkoxy radical contains up to 4 carbon atoms,
e) >=N—O—R8a,
R3a and R4a, which may be identical or different, are hydrogen, a linear or branched alkyl radical containing up to 6 carbon atoms or a phenyl radical optionally substituted with a hydroxyl or alkoxy radical containing up to 4 carbon atoms,
R5a and R6a, which may be identical or different, are chosen from hydrogen, the hydroxyl radical and linear or branched alkyl or alkoxy radicals containing up to 6 carbon atoms optionally substituted with a phenyl or indazolyl radical,
R7a is a linear or branched alkyl radical containing up to 4 carbon atoms optionally substituted with a phenyl radical or an indazolyl radical,
R8a is hydrogen or a linear or branched alkyl radical containing up to 4 carbon atoms optionally substituted with a phenyl radical,
it being understood that alk is a linear or branched alkyl radical containing up to 4 carbon atoms,
said compounds of formula (Ia) being in any possible racemic, enantiomeric or diastereoisomeric isomer form, or the addition salts with mineral and organic acids or with mineral and organic bases of said compounds of formula (Ia).

3. The compound of formula (I) as defined in claim 1, corresponding to formula (Ib): 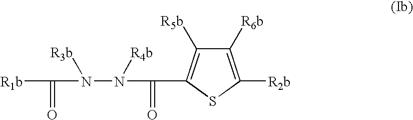
in which:
R1b is a linear or branched alkyl or alkoxy radical containing up to 6 carbon atoms substituted with a radical chosen from phenyl and fluorene radicals and an amino radical that is itself optionally substituted with a radical —C(O)—O—R7b or —C(O)—R7b, R2b is:
a) a linear or branched alkyl radical containing up to 6 carbon atoms optionally substituted with one or more radicals chosen from the radicals —NH—C(O)—O—R7b and —NH—C(O)-alk-NH—C(O)—O—R7b and phenyl, pyridyl, pyridyl, piperidyl, benzofuryl, morpholinyl, quinolyl, indolyl, benzimidazolyl and indazolyl radicals,
b) a phenyl radical optionally substituted with a linear or branched alkoxy radical containing up to 4 carbon atoms or a benzyloxy radical,
c) a naphthyl radical,
d) an amino radical optionally substituted with one or two radicals, which may be identical or different, chosen from linear or branched alkyl radicals containing up to 4 carbon atoms, radicals —C(O)-alk-NH—C(O)—O-benzyl and a benzyl radical, itself optionally substituted with an alkoxyphenoxy radical in which the alkoxy radical contains up to 4 carbon atoms,
e) >=N—O—R8b,
R3b and R4b are hydrogen,
R5b and R6b, which may be identical or different, are hydrogen, a hydroxyl radical, a linear or branched alkyl or alkoxy radical containing up to 4 carbon atoms optionally substituted with a phenyl radical,
R7b is a linear or branched alkyl radical containing up to 4 carbon atoms optionally substituted with a phenyl radical or an indazolyl radical,
R8b is hydrogen or a benzyl radical,
it being understood that alk is a linear or branched alkyl radical containing up to 4 carbon atoms,
said compounds of formula (Ib) being in any possible racemic, enantiomeric or diastereoisomeric isomer form, or the addition salts with mineral and organic acids or with mineral and organic bases of said compounds of formula (Ib).

4. The compound of formula (I), as defined in claim 1, in which R3, R4, R5, R6, are hydrogen,
said compound of formula (I), being in any possible racemic, enantiomeric or diastereoisomeric isomer form, or the addition salts with mineral and organic acids or with mineral and organic bases of said compound of formula (I).

5. The compound of formula (Ia) as defined in claim 2, in which R3a, R4a, R5a, and R6a are hydrogen, said compound of formula (Ia) being in any possible racemic, enantiomeric or diastereoisomeric isomer form., or the addition salts with mineral and organic acids or with mineral and organic bases of said compounds of formulae (Ia).

6. The compound of formula (Ib) as defined in claim 3, in which R5b, and R6b are hydrogen, said compound of formula (Ib) being in any possible racemic, enantiomeric or diastereoisomeric isomer form, or the addition salts with mineral and organic acids or with mineral and organic bases of said compounds of formulae (Ib).

7. A compound) selected from the group consisting of:
5-[(2S)-1-[[4-methyl-1-oxo-2-[[(phenylmethoxy)-carbonyl]amino]pentyl]amino]ethyl]-2-thiophenecarboxylic acid 2-[(2S)-4-methyl-1-oxo-2-[[(phenylmethoxy)carbonyl]amino]pentyl]hydrazide
5-[1-(hydroxyimino)ethyl]-2-thiophenecarboxylic acid 2-[[(9H-fluoren-9-yl)methoxy]carbonyl]hydrazide
5-[2-(phenylmethoxy)phenyl]-2-thiophenecarboxylic acid 2-[(2S)-4-methyl-1-oxo-2-[[(phenylmethoxy)-carbonyl]amino]pentyl]hydrazide
5-[[[3-(4-methoxyphenoxy)phenyl]methyl](phenylmethyl)amino]-2-thiophenecarboxylic acid 2-[(2S)-4-methyl-1-oxo-2-[[(phenylmethoxy)carbonyl]amino]pentyl]-hydrazide
5-[1-[(phenylmethoxy)imino]ethyl]-2-thiophenecarboxylic acid 2-[(2S)-4-methyl-1-oxo-2-[[(phenylmethoxy)carbonyl]amino]pentyl]hydrazide
5-[1-[[(phenylmethoxy)carbonyl]amino]ethyl]-2-thiophenecarboxylic acid 2-[[(9H-fluoren-9-yl)methoxy]-carbonyl]hydrazide
5-[1-[[(phenylmethoxy)carbonyl]amino]ethyl]-2-thiophenecarboxylic acid 2-[(2S)-4-methyl-1-oxo-2-[[(phenylmethoxy)carbonyl]amino]pentyl]hydrazide
5-[3-methyl-1-[(phenylmethoxy)carbonyl]amino]butyl]-2-thiophenecarboxylic acid 2-[(2S)-4-methyl-1-oxo-2-[[(phenylmethoxy)carbonyl]amino]pentyl]hydrazide
5-[3-methyl-1-[[(phenylmethoxy)carbonyl]amino]butyl]-2-thiophenecarboxylic acid 2-[(2S)-4-methyl-1-oxo-2-[[(phenylmethoxy)carbonyl]amino]pentyl]hydrazide isomer A, and
5-[3-methyl-1-[[(phenylmethoxy)carbonyl]amino]butyl]-2-thiophenecarboxylic acid [(2S)-4-methyl-1-oxo-2-[[(phenylmethoxy)carbonyl]amino]pentyl]hydrazide isomer B,
or the addition salts with mineral and organic acids or with mineral and organic bases thereof.

8. A process for preparing a compound of formula (I) as defined in claim 1, wherein the starting material used is a compound of formula (II): 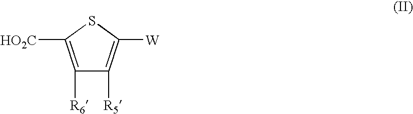
in which W is hydrogen, halogen or a nitro radical, R5′ and R6′ are as defined in claim 1, respectively, for R5 and R6, in which the optional reactive functions are optionally protected with protecting groups,
wherein compound of formula (II), depending on the value of W, may be subjected to the following reactions:
a) when W is hydrogen, the compound of formula (II) may be subjected to the reactions of route i) or ii) comprising:
route i) reacting the compound of formula (II) in which W is hydrogen with a bromo derivative of formula (III):
R2′-Br (III)
in which R2′ is as defined in claim 1 for R2 and wherein the optional reactive functions are optionally protected with protecting groups,
to give a compound of formula (IV): 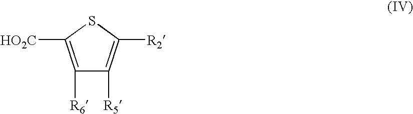
in which R2′, R5′ and R6′ have the meanings given above,
when R2′ is —CH2-phenyl, the reaction gives compound of formula (IV₁): 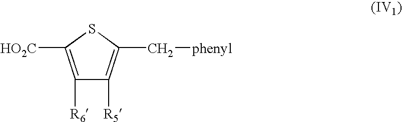
in which R5′ and R6′ have the meanings given above, and optionally, reacting compound (IV₁) in a second reaction with the compound of formula (III) to give the compound of formula (IV₂): 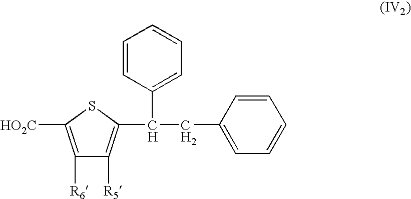
in which R5′ and R6′ are as defined above,
optionally esterifying compounds of formulae (IV), (IV₁) and (IV₂) to give the corresponding compounds of formulae (V), (V₁) and (V₂): 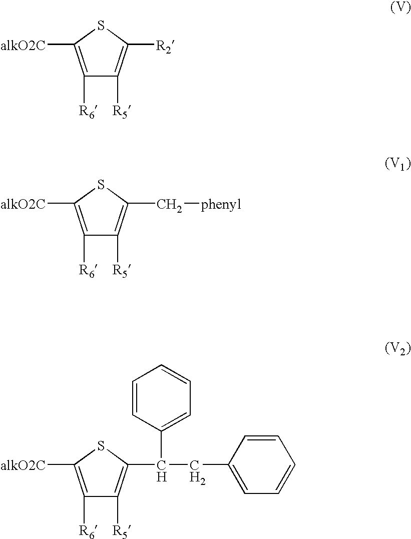
in which R2′, R5′ and R6′ have the meanings given above and alk is a linear or branched alkyl radical containing up to 4 carbon atoms,
route ii) esterifying the compound of formula (II) in which W is hydrogen by reaction with an alcohol of formula (VI):
Rc-OH (VI)
in which Rc is a linear or branched alkyl radical containing up to 6 carbon atoms,
to give compounds of formula (VII): 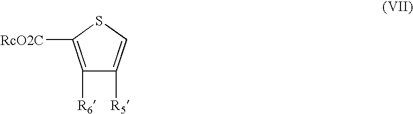
in which Rc, R5′ and R6′ have the meanings given above, reacting compounds of formula (VII) with an aldehyde of formula (VIII):
Rd-CHO (VIII)
in which Rd is hydrogen, an alkyl radical as defined above or a monocyclic radical or a radical consisting of saturated or unsaturated carbocyclic and heterocyclic fused rings that are up to 14-membered, containing one or more identical or different hetero atoms O, N, NH or S, which are themselves optionally substituted,
to give compounds of formula (IX): 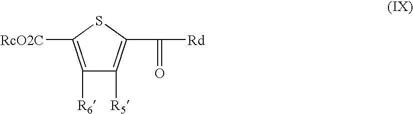
in which Rc, Rd, R5′ and R6′ have the meanings given above,
reacting compounds of formula (IX) with an amine of formula (X):
H2N—CH2—ORe (X)
in which Re is an optionally substituted arylalkyl radical,
to give compounds of formula (XI): 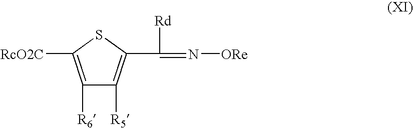
in which Rc, Rd, Re, R5′ and R6′ are as defined above, optionally selectively reducing compound of formula (XI),
to give compounds of formula (XII): 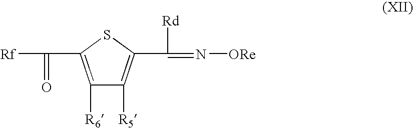
in which Rd, Re, Rf, R5′ and R6′ have the meanings given above,
optionally reducing compounds of formulae (XI) and (XII) to give the corresponding compounds of formula (XIII): 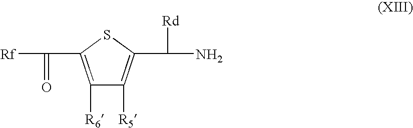
in which Rd, Rf, R5′ and R6′ have the meanings given above,
optionally saponifying compounds of formula (XIII) to give compounds of formula (XIV): 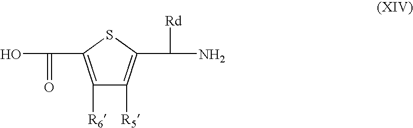
in which Rd, R5′ and R6′ have the meanings given above, optionally reacting compound of formula (XIV) with compound of formula (XV): 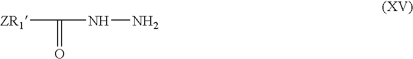
in which R1′ is as defined in claim 1 for R1 in which the optional reactive functions are optionally protected with protecting groups,
to give compounds of formula (I′x): 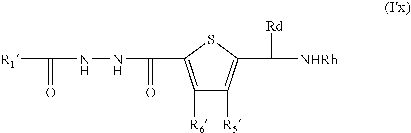
in which R1′, Rd, R5′ and R6′ have the meanings given above and Rh is —C(O)—O—R7 or —C(O)-alk-NH—C(O)—O—R7 with R7 as defined above,
b) optionally when W is halogen, reacting compound of formula (II) with an alcohol of formula (VI) as defined above, to give compounds of formula (XVI): 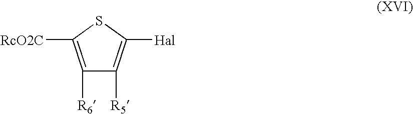
in which Rc, R5′ and R6′ have the meanings given above and Hal is halogen,
optionally reacting compound of formula (XVI) with a boron compound of formula (XVII):
R2′-B(OH)2 (XVII)
in which R2′ has the meaning given above, to give a compound of formula (XVIII): 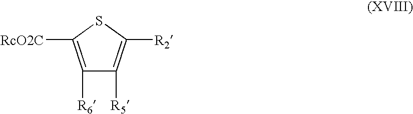
in which Rc, R2′, R5′ and R6′ have the meanings given above,
or optionally reacting a compound of formula (XVI) to with a compound of formula (XIX):
NH—RaRb (XIX)
in which Ra and Rb, which may be identical or different, are optionally substituted alkyl or aryl radical as defined above,
to give a compound of formula (XX): 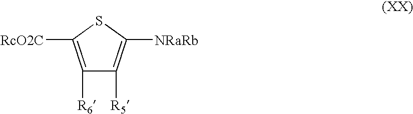
in which Rc, Ra, Rb, R5′ and R6′ are as defined above,
c) when W is a nitro radical, reacting the compound of formula (II) with an alcohol of formula (VI) as defined above, to give compounds of formula (XXI): 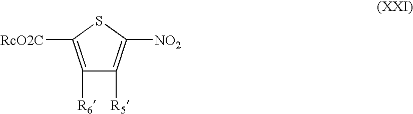
in which Rc, R5′ and R6′ have the meanings given above, optionally reacting compound (XXI) with a halo derivative of formula (XXII):
RaRbHal (XXII)
in which Ra and Rb are as defined above and Hal is halogen,
to give a compound of formula (XX) as defined above, optionally reacting compounds of formulae (IV), (IV₁), (IV₂), (V), (VI), (V₂), (scheme I) (XVIII) and (XX) as defined above with a diamine NH2-NH2 to give a compound of formula (XXIII): 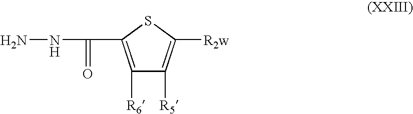
in which R5′ and R6′ have the meanings given above and R2w is —CH2-phenyl, —(CH)phenyl-CH2(phenyl), R2′ or NRaRb as defined above,
optionally reacting compound (XXIII) with a compound of formula (XXIV):
R1′-COOH (XXIV)
in which R1′ has the meaning given above, to give a compound of formula (I′y): 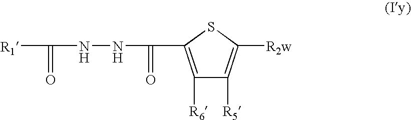
in which R1′, R2w, R5′ and R6′ have the meanings given above, and
compounds of formulae (I′x) and (I′y) may be compounds of formula (I) and in order to obtain other compounds of formula (I), said compounds of formula (I′x) and (I′y) may optionally be reacted, if desired and if necessary, to one or more of the following conversion reactions, further comprising in any order:
a) esterifying an acid function,
b) saponifying an ester function to an acid function,
c) oxidizing an alkylthio group to the corresponding sulfoxide or sulfone,
d) reacting a ketone with a hydroxylamine to form an oxime function,
e) reducing a free or esterified carboxyl function to an alcohol function,
f) cleaving an alkoxy function to a hydroxyl function, or alternatively alkylating a hydroxyl function to an alkoxy function,
g) oxidizing an alcohol function to an aldehyde, acid or ketone function,
h) converting a nitrile group to a tetrazolyl,
i) removing the protecting groups possibly borne by the protected reactive functions,
j) forming a salt by reaction with a mineral or organic acid or with a base, to give the corresponding salt,
k) resolving the racemic forms into resolved compounds, said compound of formula (I) thus obtained being in any possible racemic, enantiomeric or diastereoisomeric isomer form.

9. A pharmaceutical composition comprising a pharmaceutically acceptable carrier and an effective amount of the compound as defined in claim 1.

10. A method of treating or preventing a disease in a patient said disease associated with abnormal physiological behavior in the secretion of and/or the activity of cysteine proteases comprising administering to a patient in need thereof a therapeutically effective amount of a compound of claim 1.

11. The method as defined in claim 10 wherein the cysteine proteases are cathespins selected from the group consisting of B, H, J, L, N, S, T, C, V, W, K, O, O₂, and papain.

12. The method as defined in claim 11, wherein the cathespsin is cathepsin K.

13. The method as defined in claim 11 wherein the disease is selected from the group consisting of cartilage and bone metabolism disorders, proliferative diseases, bone cancers, cardiovascular diseases, restenosis, central nervous system diseases, immune system diseases, allergies, infectious and inflammatory diseases and autoimmune diseases.

14. The method as claimed in claim 13, whererein the disease is chosen from cartilage and bone metabolism diseases, proliferative diseases, bone cancers and neurodegenerative diseases.

15. The method of claim 14 wherein the neurodegenerative disease is Alzheimer's disease.

16. The method as claimed in claim 13, wherein the disease is chosen from cartilage and bone metabolism diseases and bone cancers.

17. The method as claimed in claim 13, wherein the disease is chosen from osteoporosis, gingival diseases including gingivitis and periodontosis, arthritis including, osteoarthritis and rheumatoid arthritis, Paget's disease, hypercalcemia and bone cancers.

18. The method as claimed in claim 17, wherein the disease to be prevented or treated is osteoporosis.
